(19) United States
(12) Patent Application Publication
(10) Pub. No.: US 2006/0106028 A1
(43) Pub. Date: May 18, 2006

(54) Polycyclic aromatics and derivatives thereof and processes for their preparation

(75) Inventors: James B. McAlpine, Montreal (CA); Arjun H. Banskota, Saint-Laurent (CA)

Correspondence Address: ECOPIA BIOSCIENCES INC., 7290 FREDERICK-BANTING, SAINT-LAURENT, QC, H4S 2A1, CA

(73) Assignee: Ecopia BioSciences, Inc.

(21) Appl. No.: 11/268,610

(22) Filed: Nov. 8, 2005

Related U.S. Application Data
(60) Provisional application No. 60/625,651, filed on Nov. 8, 2004.

(51) Int. Cl. A61K 31/496; C07D 403/02

(52) U.S. Cl. 514/253.02; 544/262

(57) ABSTRACT

This invention relates to novel biologically active polycyclic aromatics produced by cultivation of Micromonospora echinospora ssp. challisensis NRRL 12255. The invention also relates to their pharmaceutically acceptable salts, prodrugs and derivatives, and to methods of obtaining them by post-biosynthesis chemical modification. The invention further relates to the use of the polycyclic aromatics and their derivatives in the preparation of medicaments for the treatment of neoplastic conditions and bacterial infections.

### RELATED APPLICATIONS

This application claims benefit under 35 USC §119 of Provisional Application U.S. Ser. No. 60/625,651, filed Nov. 8, 2004, the entire teachings of which are incorporated herein by reference for all purposes.

### FIELD OF THE INVENTION

This invention relates to novel biologically active polycyclic aromatics, their pharmaceutically acceptable salts, prodrugs and derivatives, and to methods of obtaining them. One method for obtaining the compounds is by cultivation of Micromonospora echinospora ssp. challisensis NRRL 12255 species or a mutant or variant thereof and optional post-biosynthesis chemical modification.

### BACKGROUND OF THE INVENTION

Polyketides are a diverse class of naturally occurring molecules typically produced by a variety of organisms, including fungi and mycelial bacteria, in particular actinomycetes. Although polyketides have widely divergent structures, they are classified together because they all share a common biosynthetic scheme in which the carbon backbones of these molecules are assembled by sequential, step-wise addition of two carbon or substituted two carbon units. Polycyclic aromatics are a subclass of polyketides and comprise several fused substituted aromatic and/or quinone rings.

Polycyclic aromatics are usually found in their natural environment only in trace amounts. Moreover, due to their structural complexity, polycyclic aromatics are notoriously difficult to synthesize chemically. Nevertheless, polycyclic aromatics have been the object of research efforts from several groups for the treatment of conditions such as cancer and infectious diseases. Albofungins, simaomicins and cervinomycins, are a few examples of polycyclic aromatic molecules, which have been extensively researched. These polycyclic aromatics, all having a core backbone composed of six fused rings and bearing a γ-pyrone as E-ring, are examples of polycyclic aromatics reported to possess biological activities such as protozoacidal, antiparasitic, antifungal, antibacterial or anticancer activities (for example, see: U.S. Pat. Nos. 4,551,533; 4,649,143; 5,494,913 and 5,126,350). Another example of polycyclic aromatic recently disclosed is Echinosporamicin (see: Haiyin He et al, Helvetica Chimica Acta, Vol. 87,1385-1391 (2004); Int. Congress on Nat. Prod. Res., Aug. 4, 2004, poster 394 and U.S. Patent Application published as 2004/0220195). Echinosporamicin was reported as a Gram-positive antibacterial agent having no significant antifungal or anticancer activities. Antibiotic bravomicins were disclosed in U.S. Pat. No. 5,994,543. Another publication disclosed an antibiotic described as a mixture of polycyclic aromatic compounds associated with the mycelial-bound non-diffusible pigments of Micromonospora purpurea, NRRL 2953 (Rusnak, K. et al, Appl. Microbiol. Biotechnol., Vol. 56, 502-503 (2001)). Micromonospora echinospora ssp. challisensis (NRRL 12255) has been reported by Waitz and co-workers (U.S. Pat. No. 4,440,751) to produce the hazymicin complex, of which the two major components identified, are substituted biphenyls.

Although several biologically active polycyclic aromatics have been identified, there remains a need to obtain novel polycyclic aromatics that have enhanced properties or possess completely novel bioactivities. The complex polycyclic aromatics produced by actinomycetes are particularly valuable. In fact, because of their structural complexity, such novel polycyclic aromatics are not readily obtainable by total chemical synthesis. The present invention addresses this need by providing new polycyclic aromatic compounds with therapeutic activity.

### SUMMARY OF THE INVENTION

The invention provides polycyclic aromatics. In an aspect, the invention provides a polycyclic aromatic selected from Compounds 1 and 2, or a tautomer, or a pharmaceutically acceptable salt, solvate or prodrug thereof. In another aspect, the invention provides polycyclic aromatic analogs, which are ester, ether, amide or reduced quinone derivatives, of any one of Compound 1, Compound 2, a tautomer of any one of Compound 1 and 2, or a pharmaceutically acceptable salt or prodrug thereof.

In another aspect, the invention provides polycyclic aromatics of Formula I or II, as illustrated below, which includes Compounds 1 and 2 and their analogs obtainable by chemical modification, or a tautomer, or a pharmaceutically acceptable salt, solvate or prodrug thereof. In one embodiment, the polycyclic aromatic is selected from Compounds 1 to 35 as described herein, or a tautomer of any one of Compounds 1 to 35; or pharmaceutically acceptable salts or prodrugs thereof.

The invention further provides a polycyclic aromatic of Formula I or II obtained by a method comprising cultivating a Micromonospora strain under aerobic conditions in a nutrient medium comprising at least one source of carbon atoms and at least one source of nitrogen atoms, and isolating a polycyclic aromatic from the cultivated bacteria. In another aspect of the invention, the compound obtained from cultivation and isolation described above is further chemically modified. In one embodiment, the strain is a Micromonospora echinospora species or a mutant thereof. In another embodiment, the strain is a Micromonospora echinospora ssp. challisensis or a mutant thereof. In another embodiment, the strain is Micromonospora echinospora ssp. challisensis having accession number NRRL 12255. In a further embodiment, the polycyclic aromatic generates a ¹H NMR spectra essentially as detailed in Table 3. In a further embodiment, the polycyclic aromatic is selected from Compound 1 and Compound 2. In a further embodiment, the nutrient medium is selected from the media of Table 1. In a further embodiment, the cultivation is carried out under aerobic conditions. In a further embodiment, the cultivation is carried out at a temperature ranging from about 18° C. to about 40° C., preferably between 18° C. and 30° C. In a further embodiment, the cultivation is carried out at a pH ranging from about 6 to about 9.

The invention further provides a process for producing a polycyclic aromatic of Formula I or II, comprising cultivating a Micromonospora strain in a nutrient medium comprising at least one source of carbon atoms and at least one source of nitrogen atoms, and isolating and purifying the polycyclic aromatic. In another aspect of the invention, the process further comprises step of chemical modifying the isolated and purified polycyclic aromatic. In one embodiment, the strain is a Micromonospora echinospora species. In another embodiment, the strain is a Micromonospora echinospora ssp. challisensis strain or a mutant thereof. In a further embodiment, the strain is Micromonospora echinospora ssp. challisensis having accession number NRRL 12255. In a further embodiment, the carbon and nitrogen source is selected from the components of Table 1. In a further embodiment, the nutrient medium is selected from the media of Table 1. In a further embodiment, the cultivation is carried out under aerobic conditions. In a further embodiment, the cultivation is carried out at a temperature ranging from about 18° C. to about 40° C., preferably between 18° C. and 30° C. In a further embodiment, the cultivation is carried out at a pH ranging from about 6 to about 9. In a further embodiment, the chemical modification step involves at least a chemical reaction selected from: esterification, etherification, amide formation and quinone reduction.

The invention further provides polycyclic aromatics of Formula I or Formula II that are derivatives or structural analogs of any of Compounds 1 or 2. In one embodiment the polycyclic aromatics of Formula I are produced by post-biosynthesis chemical modification of any of Compounds 1 or 2. In another embodiment of the invention, the compounds of Formula I or Formula II are esters, ethers and amides of any one of: Compound 1, Compound 2, or a tautomer of any one of Compound 1 or 2.

The invention further provides a process for the preparation of a compound of Formula I or Formula II, comprising the step of chemically modifying a compound selected from Compound 1 and Compound 2, and optionally isolating the modified compound. In one embodiment, the modification comprises an esterification step. In another embodiment, the modification comprises an etherification step. In another embodiment, the modification comprises an amide formation step. In another embodiment, the modification comprises a quinone reduction step. In a further embodiment, the modification process comprises a single chemical modification step. In a further embodiment, the modification process comprises two or more chemical modification steps. In a further embodiment, the modification step comprises three or more chemical modification steps.

The invention further relates to pharmaceutical compositions comprising a compound of Formula I or Formula II, or a pharmaceutically acceptable salt or prodrug thereof, together with a pharmaceutically acceptable carrier. In one embodiment, the compound is selected from Compounds 1 to 35, or a pharmaceutically acceptable salt, solvate or prodrug thereof. In another embodiment, the compound is selected from Compound 1 and Compound 2, or a pharmaceutically acceptable salt, solvate or prodrug thereof.

The invention further provides a compound selected from Compound 1, Compound 2, a compound of Formula I or Formula II, or a pharmaceutically acceptable salt or prodrug thereof, for use as an antineoplastic agent. The invention further provides a compound selected from Compound 1, Compound 2, a compound of Formula I, or a pharmaceutically acceptable salt or prodrug thereof, for use as an antibacterial agent. The invention further provides a compound selected from Compound 1, Compound 2, a compound of Formula I or Formula II, or a pharmaceutically acceptable salt or prodrug thereof, for use as an antifungal agent.

The invention further provides use of a compound of Formula I or Formula II, or a tautomer, or a pharmaceutically acceptable salt or prodrug thereof, as an antineoplastic, antibacterial or antifungal agent. In one embodiment, the compound is selected from Compounds 1 to 35, or a pharmaceutically acceptable salt, solvate or prodrug thereof. In another embodiment, the compound is selected from Compound 1 and Compound 2, or a pharmaceutically acceptable salt, solvate or prodrug thereof.

The invention further provides use of a compound of Formula I or Formula II, or a tautomer, or a pharmaceutically acceptable salt or prodrug thereof, in the preparation of a medicament for the treatment of a neoplastic condition, or a bacterial or fungal infection. In one embodiment, the compound is selected from Compounds 1 to 35, or a pharmaceutically acceptable salt, solvate or prodrug thereof. In another embodiment, the compound is selected from Compound 1 and Compound 2, or a pharmaceutically acceptable salt, solvate or prodrug thereof.

The invention further provides a commercial package comprising a compound of Formula I or Formula II, or a tautomer, or a pharmaceutically acceptable salt or prodrug thereof, and a written matter describing instructions for the use of the compound for treating a neoplastic condition, or a bacterial or fungal infection. In one embodiment, the compound is selected from Compounds 1 to 35, or a pharmaceutically acceptable salt, solvate or prodrug thereof. In another embodiment, the compound is selected from Compound 1 and Compound 2, or a pharmaceutically acceptable salt, solvate or prodrug thereof.

The invention also provides methods of inhibiting bacterial cell growth, which comprise contacting said bacterial cell with a compound of Formula I or Formula II, or a pharmaceutically acceptable salt or prodrug thereof. The invention further encompasses methods for treating a bacterial infection in a subject, comprising administering to said subject suffering from said bacterial infection, a therapeutically effective amount of a compound of Formula I or Formula II, or a pharmaceutically acceptable salt or prodrug thereof. In one embodiment, the compound is selected from Compounds 1 to 35, or a pharmaceutically acceptable salt or prodrug thereof. In another embodiment, the compound is Compound 1 or 2, or a pharmaceutically acceptable salt or prodrug thereof.

In one embodiment, the bacterial infection or organism involved in any of the above-mentioned uses and methods, is selected from: Streptococcus pneumoniae, Streptococcus pyogenes, Enterococcus faecalis, Enterococcus faecium, Klebsiella pneumoniae, Enterobacter spp., Proteus spp., Pseudomonas aeruginosa, Serratia marcescens, Staphylococcus aureus, Coagulase negative Staphylococcus, Haemophilus infuenzae, Bacillus anthracis, Mycoplasma pneumoniae, and Staphylococcus epidermidis.

In still further aspects, the invention relates to methods of inhibiting the growth of a cancer cell by contacting the cancer cell with a polycyclic aromatic of Formula I or Formula II, or a pharmaceutically acceptable salt or prodrug thereof, and inhibiting the growth of a cancer cell in a mammal by administering the compound to the mammal. The invention further relates to methods of treating a neoplasm, or a pre-cancerous or cancerous condition in a subject, by administering a therapeutically effective amount of a polycyclic aromatic to a subject in need thereof. In one embodiment, the compound is selected from Compounds 1 to 35, or a pharmaceutically acceptable salt or prodrug thereof. In another embodiment, the compound is Compound 1 or Compound 2, or a pharmaceutically acceptable salt or prodrug thereof.

In one embodiment, the neoplastic cell or condition involved in any of the above-mentioned uses and methods, is selected from: leukemia, melanoma, breast cancer, lung cancer, pancreatic cancer, ovarian cancer, renal cancer, colon or colorectal cancer, prostate cancer, and CNS cancer. In another embodiment, the cancer cell, and pre-cancerous or cancerous condition, in the above-mentioned methods and uses, is selected from leukemia, breast cancer, prostate cancer, and CNS cancer.

The invention also provides methods of inhibiting fungal cell growth, which comprise contacting said fungal cell with a compound of Formula I or Formula II, or a pharmaceutically acceptable salt or prodrug thereof. The invention further encompasses methods for treating a fungal infection in a subject, comprising administering to said subject suffering from said fungal infection, a therapeutically effective amount of a compound of Formula I or Formula II, or a pharmaceutically acceptable salt or prodrug thereof. In one embodiment, the compound is selected from Compounds 1 to 35, or a pharmaceutically acceptable salt or prodrug thereof. In another embodiment, the compound is Compound 1 or Compound 2, or a pharmaceutically acceptable salt or prodrug thereof.

In one embodiment, the fungal infection or organism involved in any of the above-mentioned uses and methods, is selected from: Candida species, S. cerevisiae; Aspergillus species; Fusarium spp.; Scedosporium spp.; Cryptococcus spp.; Mucor ssp.; Histoplasma spp.; Trichosporon spp.; or Blastomyces spp. In another embodiment, the fungal infection or organism involved is a Saccharomyces cerevisiae species.

### BRIEF DESCRIPTION OF THE FIGURES

FIG. 1: shows the mean (±SD) plasma concentrations of Compound 1 in Swiss mice following 25 mg/kg intravenous (iv) and intraperitoneal (ip) bolus administrations.

### DETAILED DESCRIPTION

The present invention relates to novel polycyclic aromatics, exemplified herein as Compounds 1 and 2, which are isolated from strains of actinomycetes, Micromonospora sp. such as Micromonospora echinospora challisensis NRRL 12255, or a mutant or variant thereof.

The invention also relates to polycyclic aromatics of Formula I or Formula II, a new class of polycyclic aromatics represented by Compounds 1 and 2 and their structural analogs produced by chemical modification, using techniques described herein and well known to those skilled in the synthesis of natural products. The invention further relates to tautomeric forms, and to pharmaceutically acceptable salts, solvates and prodrugs of Compounds 1 and 2, and the compounds of Formula I or Formula II.

The present invention also relates to pharmaceutical compositions comprising a polycyclic aromatic of the invention. In an aspect of this invention, polycyclic aromatics of the invention are useful as cytotoxic agents, and for use as inhibitors of neoplastic, bacterial and fungal cell growth.

Accordingly, the present invention relates to methods of using the compounds and compositions to inhibit bacterial growth, and methods of using the compounds and pharmaceutical compositions comprising same, to treat diseases, including neoplastic conditions, and bacterial or fungal infections.

### I. DEFINITIONS

Certain terms, when used in this application, have their common meaning unless otherwise specified. For convenience, the meaning of some terms and phrases used in the specification, examples, and appended claims, are provided below.

As used herein, abbreviations have their common meaning. Unless otherwise noted, the abbreviations “Ac”, “Me”, “Et”, “Pr”, “i-Pr”, “Bu”, “Bz”, “Bn” and “Ph”, respectively refer to acetyl, methyl, ethyl, propyl (n- or iso-propyl), iso-propyl, butyl (n-, iso-, sec- or tert-butyl), benzoyl, benzyl and phenyl. Abbreviations in the specification correspond to units of measure, techniques, or properties as follows: “RT” and “Rt” mean retention time, “min” means minutes, “h” means hour(s), “μL” means microliter(s), “mL” means milliliter(s), “mM” means millimolar, “M” means molar, “mmole” means millimole(s), “eq” means molar equivalent(s). “High Pressure Liquid Chromatography” or “High Performance Liquid Chromatography” are abbreviated HPLC.

The term “polycyclic aromatics” of the invention refers to the compounds of Formula I or Formula II, exemplified by Compounds 1 and 2, produced by fermentation, and by structural analogs or semisynthetic derivatives produced by chemical modification of Compound 1 or Compound 2, and to tautomers and pharmaceutically acceptable salts, solvates and prodrugs thereof.

The terms “analogs”, “derivative” or “semisynthetic derivative” refer to chemical compounds that are structurally similar to Compound 1 or 2 but differ slightly in composition (Merriam-Webster's Collegiate Dictionary, 10^{th} edition, 1998). Herein the terms refer to the polycyclic aromatic compounds of Formula I or Formula II, or pharmaceutically acceptable salts or prodrugs thereof, produced by chemical modification of Compound 1 or Compound 2, and exemplified by Compounds 3 to 35. The derivative is obtained by one or more chemical modification steps. The derivative is optionally further modified, if necessary, by known methods such as hydrolysis, oxidation, reduction, deprotection.

The term “chemical modification” includes esterification, etherification, and amide formation to produce respectively esters, ethers and amides. The term further includes ester hydrolysis, demethylation and quinone reduction reactions.

The term “ether” refers to a compound obtained by the replacement of a hydrogen atom on one or more oxygen atom from an alcohol by an R⁹ replacement group. Ethers are produced by O-alkylation (or etherification) reactions as defined in Scheme 2(a).

The term “ester” refers to a compound obtained by the replacement of a hydrogen atom on at least one oxygen atom from alcohols by an R¹⁰C(O) group. The term further encompasses ester analogs including, without limitation, carbonate, carbamate, guanidino, and the like. The term “ester” equally includes a compound obtained by the replacement of a hydrogen atom of a carboxylic acid by an R⁵ replacement group. Esters are produced by O-acylation (esterification) reactions as defined in Scheme 2(b), and esterification reactions as defined in Scheme 1(a).

The term “amide” refers to a compound obtained by the replacement of the OH of a carboxylic acid (C(O)OH) by an amine (R⁶R⁷N) replacement group. Amides are produced by amidation reactions as defined in Scheme 1(c).

The term “demethylation” refers to the cleavage of the methyl group of a methoxy. Demethylation products are produced as described in Scheme 3.

The term “quinone reduction” refers to the reduction of the quinone of ring B and/or ring E moiety by methods such as described in Schemes 4(a) and (b).

The terms “tautomers” and “tautomeric forms” refer to compounds, which are in rapid equilibrium between two or more structurally distinct compounds. The compounds of the invention are useful as a single or as an equilibrium mixture of the different forms present (J. March, “Advanced Organic Chemistry”, 4^{th} Edition, John Wiley & Sons, New York (1992), pages 69-74). Examples of tautomers include, without limitation, proton-shift tautomerism such as double-bonds migrations, keto-phenol and keto-enol tautomerism. For example, Compounds 33 and 34 may be considered tautomers of one another. Other examples of tautomers of the compounds of the invention further include tautomers of Compounds 29 to 32, wherein B and/or E ring is a phenol ring, which may be in equilibrium with its “cyclohexadienone” form (see Scheme 4(a)).

The term “alkyl” refers to linear or branched, saturated hydrocarbon groups. Examples of saturated alkyl groups include, without limitation, methyl, ethyl, n-propyl, isopropyl, sec-butyl, iso-butyl, n-butyl, pentyl, isoamyl, hexyl, heptyl, and the like. Alkyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl, oxo, guanidino and formyl.

The term “C₁₋ₙalkyl”, wherein n is an integer from 2 to 12, refers to an alkyl group having from 1 to the indicated “n” number of carbons. The C₁₋ₙalkyl can be a straight or branched chain.

The term “alkenyl” refers to linear or branched hydrocarbon groups having from one to three carbon-carbon double bonds. Examples of alkenyl groups include, without limitation, vinyl, 1-propene-2-yl, 1-butene-4-yl, 2-butene-4-yl, 1-pentene-5-yl and the like. Alkenyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl, formyl, oxo and guanidino. The double bond portion(s) of the unsaturated hydrocarbon chain may be either in cis or trans configuration.

The term “C₂₋ₙalkenyl”, wherein n is an integer from 3 to 12, refers to an alkenyl group having from 2 to the indicated “n” number of carbons. The C₂₋ₙalkenyl can be a straight, cyclic or branched chain.

The term “alkynyl” refers to linear or branched hydrocarbon groups having at least one carbon-carbon triple bond. Examples of alkynyl groups include, without limitation, ethynyl, 1-propyne-3-yl, 1-butyne-4-yl, 2-butyne-4-yl, 1-pentyne-5-yl and the like. Alkynyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl, formyl, oxo and guanidine.

The term “C₂₋ₙalkynyl”, wherein n is an integer from 3 to 12, refers to an alkynyl group having from 2 to the indicated “n” number of carbons. The C₂₋ₙalkynyl can be a straight or branched chain.

The term “cycloalkyl” or “cycloalkyl ring” refers to cyclic hydrocarbon groups comprising a saturated or partially unsaturated (non-aromatic) carbocyclic ring in a single or fused carbocyclic ring system having from three to fifteen ring members. Examples of cycloalkyl groups include, without limitation, cyclopropyl, cyclobutyl, cyclopentyl, cyclohexyl, cycloheptyl, cyclopentene, and cyclohexene. Cycloalkyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl and formyl.

The term “C₃₋ₙcycloalkyl”, wherein n is an integer from 4 to 15, refers to a cycloalkyl group having from 3 to the indicated “n” number of carbons.

The term “heterocycloalkyl”, “heterocyclic” or “heterocycloalkyl ring” refers to a cycloalkyl group, as defined above, fuether containing one to four hetero atoms or hetero groups selected from O, N, NH, NRX, PO₂, S, SO or SO₂ in a single or fused heterocyclic ring system having from three to fifteen ring members. Examples of a heterocycloalkyl, heterocyclic or heterocycloalkyl ring include, without limitation, pyrrolidino, tetrahydrofuranyl, dihydrofuran, tetrahydrodithienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3,1,0]hexanyl, 3-azabicyclo[4,1,0]heptanyl, 3H-indolyl, quinolizinyl, and the like. The foregoing heterocycloalkyl groups, as derived from the compounds listed above, may be C-attached or N-attached where such is possible. Heterocycloalkyl, heterocyclic or heterocycloalkyl ring may optionally be substituted with substituents selected from acyl, amino, acylamino, acyloxy, oxo, thiocarbonyl, imino, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl and formyl.

The term “C₃₋ₙheterocycloalkyl”, wherein n is an integer from 4 to 15, refers to an heterocycloalkyl group having from 3 to the indicated “n” number of atoms in the cycle and at least one hetero group as defined above.

The term “aryl” or “aryl ring” refers to common aromatic groups having “4n+2” π electrons, wherein n is an integer from 1 to 3, in a conjugated monocyclic or polycyclic system and having from six to fourteen ring atoms. Aryl may be directly attached, or connected via a C₁₋₃alkyl group (also referred to as aralkyl). Examples of aryl include, without limitation, phenyl, benzyl, phenethyl, 1-phenylethyl, tolyl, naphthyl, biphenyl, terphenyl groups, and the like. Aryl may optionally be substituted with one or more substituent group selected from acyl, amino, acylamino, acyloxy, azido, alkythio, carboalkoxy, carboxy, carboxyamido, cyano, halo, hydroxyl, nitro, thio, alkyl, alkenyl, alkynyl, cycloalkyl, heterocyclyl, aryl, heteroaryl, alkoxy, aryloxy, sulfinyl, sulfonyl and formyl.

The term “C₅₋ₙaryl”, wherein n is an integer from 5 to 14, refers to an aryl group having from 5 to the indicated “n” number of atoms, including carbon, nitrogen, oxygen and sulfur. The C₅₋ₙaryl can be mono or polycyclic.

The term “heteroaryl” or “heteroaryl ring” refer to aryl rings, as defined above, further containing one to four heteroatoms selected from oxygen, nitrogen, sulphur or phosphorus. Examples of heteroaryl include, without limitation, pyridyl, imidazolyl, pyrimidinyl, pyrazolyl, triazolyl, tetrazolyl, furyl, thienyl, isooxazolyl, thiazolyl, oxazolyl, isothiazolyl, pyrrollyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl groups. Heteroaryl may optionally be substituted with one or more substituent group selected from acyl, amino, acylamino, acyloxy, azido, alkythio, carboalkoxy, carboxy, carboxyamido, cyano, halo, hydroxyl, nitro, thio, alkyl, alkenyl, alkynyl, cycloalkyl, heterocyclyl, aryl, heteroaryl, alkoxy, aryloxy, sulfinyl, sulfonyl and formyl. Heteroaryl may be directly attached, or connected via a C₁₋₃alkyl group (also referred to as heteroaralkyl). The foregoing heteroaryl groups, as derived from the compounds listed above, may be C-attached or N-attached where such is possible.

The term “C₅₋ₙheteroaryl”, wherein n is an integer from 5 to 14, refers to an heteroaryl group having from 5 to the indicated “n” number of atoms, including carbon, nitrogen, oxygen and sulphur atoms. The C₅₋ₙaryl can be mono or polycyclic.

The terms “halo” or “halogen” refer to bromine, chlorine, fluorine or iodine substituents.

The term “amino acid” refers to an organic acid containing an amino group. The term includes both naturally occurring and synthetic amino acids; therefore, the amino group can be but is not required to be, attached to the carbon next to the acid. A “C-coupled amino acid” substituent is attached to the heteroatom (phenolic oxygen) of the parent molecule via its carboxylic acid function and forms an ester with the parent molecule. An “N-coupled amino acid” substituent is attached to the carboxylic acid of the parent molecule via its amine function and forms an amide with the parent molecule. Examples of amino acids include, without limitation, alanine, valine, leucine, isoleucine, proline, phenylalanine, tryptophane, methionine, glycine, serine, threonine, cysteine, asparagines, glutamine, tyrosine, histidine, lysine, arginine, aspartic acid, glutamic acid, desmosine, ornithine, 2-aminobutyric acid, cyclohexylalanine, dimethylglycine, phenylglycine, norvaline, norleucine, hydroxylysine, allo-hydroxylysine, hydroxyproline, isodesmosine, allo-isoleucine, ethylglycine, beta-alanine, aminoadipic acid, aminobutyric acid, ethyl asparagine, and N-methyl amino acids. Amino acids can be pure L or D isomers or mixtures of L and D isomers.

The compounds of the present invention can possess one or more asymmetric carbon atoms and can exist as optical isomers forming mixtures of racemic or non-racemic compounds. The compounds of the present invention are useful as single isomers or as a mixture of stereochemical isomeric forms. Diastereoisomers, i.e., nonsuperimposable stereochemical isomers, can be separated by conventional means such as chromatography, distillation, crystallization or sublimation. The optical isomers can be obtained by resolution of the racemic mixtures according to conventional processes, including chiral chromatography (e.g. HPLC), immunoassay techniques, or the use of covalently (e.g. Mosher's esters) or non-covalently (e.g. chiral salts) bound chiral reagents to respectively form a diastereomeric ester or salt, which can be further separated by conventional methods, such as chromatography, distillation, crystallization or sublimation. The diastereomeric ester or salt is then cleaved or exchanged by conventional means, to recover the desired optical isomer(s).

The invention encompasses isolated or purified compounds. An “isolated” or “purified” compound refers to a compound which represents at least 10%, 20%, 50%, 80% or 90% of the compound of the present invention present in a mixture, provided that the mixture comprising the compound of the invention has demonstrable (i.e. statistically significant) biological activity such as cytotoxic activity when tested in conventional biological assays known to a person skilled in the art.

The term “pharmaceutically acceptable salt” refers to nontoxic salts synthesized from a compound which contains a basic or acidic moiety by conventional chemical methods. Generally, such salts can be prepared by reacting the free acid or base forms of these compounds with a stoechiometric amount of the appropriate base or acid in water or in an organic solvent, or in a mixture of the two; generally, nonaqueous media like ether, ethyl acetate, methanol, ethanol, isopropanol, or acetonitrile are preferred. Another method for the preparation of salts is by the use of ion exchange resins. The term “pharmaceutically acceptable salt” includes both acid addition salts and base addition salts, either of the parent compound or of a prodrug or solvate thereof. The nature of the salt is not critical, provided that it is pharmaceutically acceptable. Exemplary acids used in acid addition salts include, without limitation, hydrochloric, hydrobromic, hydroiodic, nitric, carbonic, sulfuric, sulfonic, phosphoric, formic, acetic, citric, tartaric, succinic, oxalic, malic, glutamic, propionic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactaric, galacturonic acid and the like. Suitable pharmaceutically acceptable base addition salts include, without limitation, metallic salts made from aluminium, calcium, lithium, magnesium, potassium, sodium and zinc or organic salts made from N,N′-dibenzylethylenediamine, chloroprocaine, choline, diethanolamine, ethylenediamine, N-methylglucamine, lysine, procaine and the like. Additional examples of pharmaceutically acceptable salts are listed in Berge et al (1977), Journal of Pharmaceutical Sciences, vol 66, no 1, pp 1-19. Preferred salts of Compound 1 are base addition salts, such as base addition salts of the carboxylic acid of position 24 (31-OH becomes 31-O⁻M⁺, wherin M may be, for example, sodium, potassium, ammonium, and the like) (see Example 3 for atom numbering).

The term “solvate” refers to a physical association of a compound of this invention with one or more solvent molecules, whether organic or inorganic. This physical association may include hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, and the like.

The term “pharmaceutically acceptable prodrug” means any pharmaceutically acceptable ester, salt of an ester or any other derivative of a compound of this invention, which upon administration to a subject, is capable of providing, either directly or indirectly, a compound of this invention or a biologically active metabolite or residue thereof. Particularly favored salts or prodrugs are those with improved properties, such as solubility, efficacy, or bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. As used herein, a prodrug is a drug having one or more functional groups covalently bound to a carrier wherein metabolic release of the drug occurs in vivo when the drug is administered to a mammalian subject. Pharmaceutically acceptable prodrugs of the compounds of this invention include derivatives of hydroxyl, carboxylic acids and amino groups such as, without limitation, acyloxymethyl, acyloxyethyl and acylthioethyl ethers, esters, amino acid esters, phosphate esters, sulfonate and sulfate esters, and metal salts, and the like.

### II. COMPOUND OF THE INVENTION

In one aspect of this embodiment the invention relates to novel polycyclic aromatics, referred to herein as Compound 1 and Compound 2: 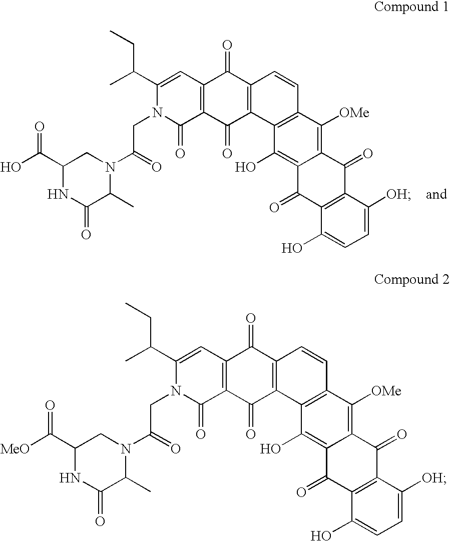
or tautomers, or pharmaceutically acceptable salts, solvates and prodrugs thereof.

Compounds 1 and 2 may be characterized by any one or more of their physicochemical and spectral properties given below, such as their mass, UV, and NMR spectroscopic data.

In an aspect, the invention provides an ester, ether or amide derivative of any one of: Compound 1, Compound 2, a tautomer of any one of Compounds 1 or 2, or a pharmaceutically acceptable salt, solvate or prodrug thereof.

In another aspect the invention relates to derivatives of any one of Compounds 1 and 2, as represented by the polycyclic aromatics of Formula I: 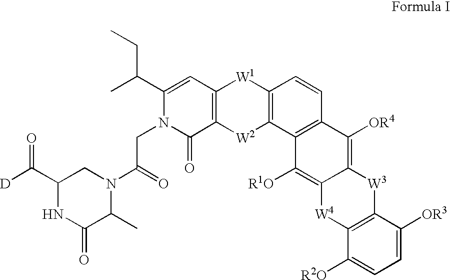
wherein,

D is selected from the group consisting of OH, —OR⁵ and —NR⁶R⁷;

W¹ and W² are each C(O); or W¹ and W² are each C(OR⁸) when taken together with their adjacent carbon atoms to form an aromatic ring; or one of W¹ and W² is C(OR⁸) and the other is C(H) when taken together with their adjacent carbon atoms to form an aromatic ring;

W³ and W⁴ are each C(O); or W³ and W⁴ are each C(OR⁸) when taken together with their adjacent carbon atoms to form an aromatic ring; or one of W³ and W⁴ is C(OR⁸) and the other is C(H) when taken together with their adjacent carbon atoms to form an aromatic ring;

R¹, R², R³, R⁴ and R⁸ are each independently selected from H, R⁹ and C(O)R¹⁰;

R⁵ is selected from the group consisting of C₁₋₁₀alkyl, C₂₋₁₀alkenyl, C₂₋₁₀alkynyl, C₃₋₁₀cycloalkyl, C₃₋₁₀heterocycloalkyl, C₅₋₁₀aryl and C₅₋₁₀heteroaryl;

R⁶ is selected from the group consisting of H, C₁₋₁₀alkyl, C₂₋₁₀alkenyl and C₂₋₁₀alkynyl;

R⁷ is selected from the group consisting of H, C₁₋₁₀alkyl, C₂₋₁₀alkenyl, C₂₋₁₀alkynyl, C₅₋₁₀aryl, C₅₋₁₀heteroaryl, C₃₋₁₀cycloalkyl and C₃₋₁₀heterocycloalkyl; or the group is an N-coupled amino acid;

R⁹ is selected from the group consisting of C₁₋₁₀alkyl, C₂₋₁₀alkenyl, C₂₋₁₀alkynyl, C₃₋₁₀cycloalkyl and C₃₋₁₀heterocycloalkyl;

R¹⁰ is selected from the group consisting of H, C₁₋₁₀alkyl, C₂₋₁₀alkenyl, C₂₋₁₀alkynyl, C₅₋₁₀aryl, C₅₋₁₀heteroaryl, C₃₋₁₀cycloalkyl and C₃₋₁₀heterocycloalkyl; or —C(O)R¹⁰ is a C-coupled amino acid;

wherein, when any of R¹, R², R³, R⁴, R⁵, R⁶, R⁷, R⁸, R⁹ and R¹⁰ comprises an alkyl, alkenyl, alkynyl, aryl, heteroaryl, cycloalkyl, or heterocycloalkyl group, then the alkyl, alkenyl, alkynyl, aryl, heteroaryl, cycloalkyl, or heterocycloalkyl group is optionally substituted with substituents selected from acyl, amino, acylamino, acyloxy, carboalkoxy, carboxy, carboxyamido, cyano, halo, hydroxyl, nitro, thio, C₁₋₆alkyl, C₂₋₇alkenyl, C₂₋₇alkynyl, C₃₋₁₀cycloalkyl, C₃₋₁₀heterocycloalkyl, C₆₋₁₀aryl, C₅₋₁₀heteroaryl, alkoxy, aryloxy, sulfinyl, sulfonyl, oxo, guanidino and formyl;

or a tautomer, or a pharmaceutically acceptable salt, solvate or prodrug thereof.

In another aspect the invention relates to derivatives of any one of Compounds 1 and 2, as represented by the polycyclic aromatics of Formula II: 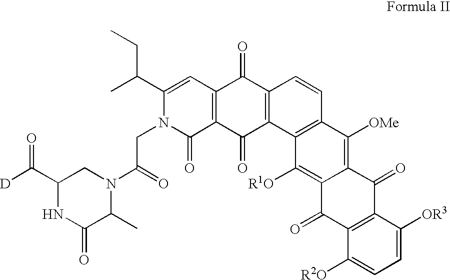
wherein,

D is selected from the group consisting of OH, —OR⁵ and —NR⁶R⁷;

R¹, R² and R³ are each independently selected from H, R⁹ and C(O)R¹⁰;

R⁵ is selected from the group consisting of C₁₋₁₀alkyl, C₂₋₁₀alkenyl, C₂₋₁₀alkynyl, C₃₋₁₀cycloalkyl, C₃₋₁₀heterocycloalkyl, C₅₋₁₀aryl and C₅₋₁₀heteroaryl;

R⁶ is selected from the group consisting of H, C₁₋₁₀alkyl, C₂₋₁₀alkenyl and C₂₋₁₀alkynyl;

R⁷ is selected from the group consisting of C₁₋₆alkyl, C₅₋₁₀aryl, C₅₋₁₀heteroaryl, C₃₋₁₀cycloalkyl and C₃₋₁₀heterocycloalkyl; or the group —NR⁶R⁷ is an N-coupled amino acid;

R⁹ is selected from the group consisting of C₁₋₁₀alkyl, C₂₋₁₀alkenyl, C₂₋₁₀alkynyl, C₃₋₁₀cycloalkyl and C₃₋₁₀heterocycloalkyl;

R¹⁰ is selected from the group consisting of H, C₁₋₁₀alkyl, C₂₋₁₀alkenyl, C₂₋₁₀alkynyl, C₅₋₁₀aryl, C₅₋₁₀heteroaryl, C₃₋₁₀cycloalkyl and C₃₋₁₀heterocycloalkyl; or —C(O)R¹⁰ is a C-coupled amino acid

wherein, when any of R¹, R², R³, R⁵, R⁶, R⁷, R⁹ and R¹⁰ comprises an alkyl, alkenyl, alkynyl, aryl, heteroaryl, cycloalkyl, or heterocycloalkyl group, then the alkyl, alkenyl, alkynyl, aryl, heteroaryl, cycloalkyl, or heterocycloalkyl group is optionally substituted with substituents selected from acyl, amino, acylamino, acyloxy, carboalkoxy, carboxy, carboxyamido, cyano, halo, hydroxyl, nitro, thio, C₁₋₆alkyl, C₂₋₇alkenyl, C₂₋₇alkynyl, C₃₋₁₀cycloalkyl, C₃₋₁₀heterocycloalkyl, C₆₋₁₀aryl, C₅₋₁₀heteroaryl, alkoxy, aryloxy, sulfinyl, sulfonyl, oxo, guanidino and formyl;

or a tautomer, or a pharmaceutically acceptable salt or prodrug thereof.

In one embodiment, R¹, R² and R³ are each H, and all other groups are as previously defined. In a further embodiment, one of R¹, R² and R³ is C₁₋₆alkyl and the others are each H, and all other groups are as previously defined. In a further embodiment, two of R¹, R² and R³ are C₁₋₆alkyl and the other is H, and all other groups are as previously defined. In a further embodiment, R¹, R² and R³ are each C₁₋₆alkyl, and all other groups are as previously defined. In a further embodiment, at least one of R¹, R² and R³ is C(O)C₁₋₆alkyl, and all other groups are as previously defined. In a further embodiment, D is OH; and all other groups are as previously defined. In a further embodiment, D is OCH₃; and all other group are as previously defined. In a further embodiment, D is OC₁₋₆alkyl; and all other group are as previously defined. In a further embodiment, D is NHC₁₋₆alkyl; and all other group are as previously defined. In a further embodiment, D is NH(C₁₋₆alkyl)₂; and all other group are as previously defined. In a further embodiment, D is an N-coupled natural amino acid; and all other group are as previously defined. In yet a further embodiment, the invention provides an ether, ester or amide, or a pharmaceutically acceptable salt, solvate or prodrug of any one of the foregoing compound.

The following are exemplary compounds of the invention: 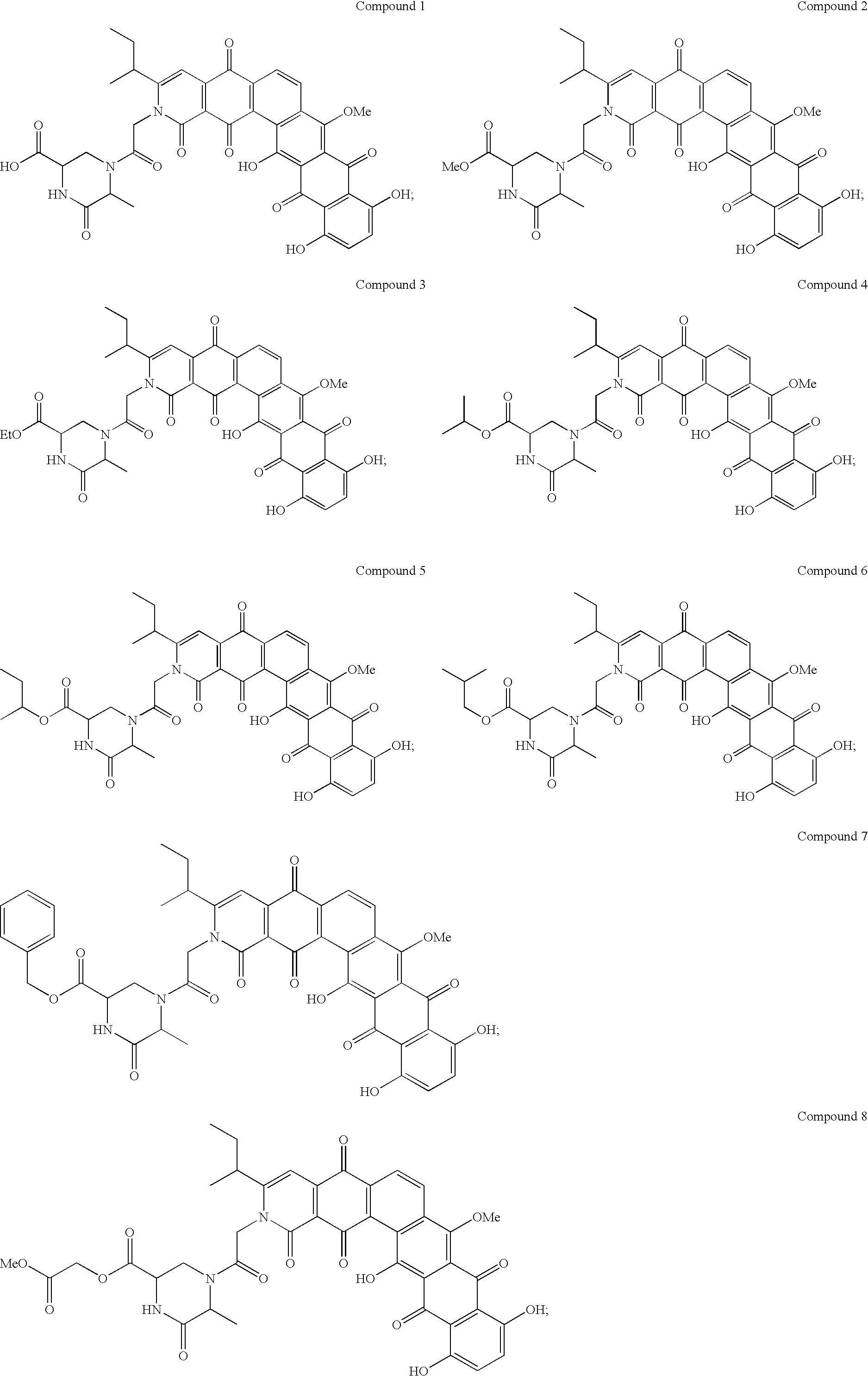 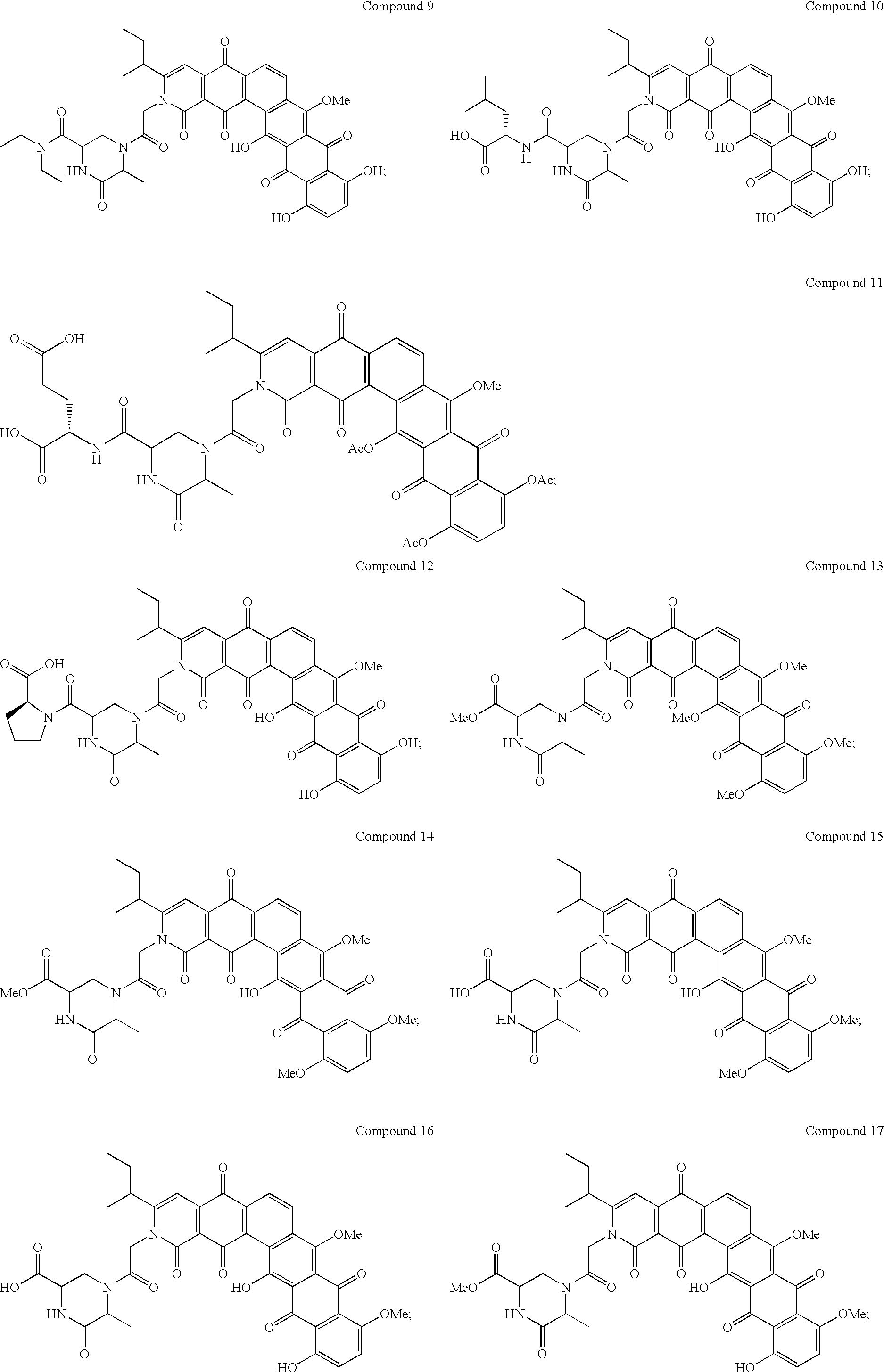 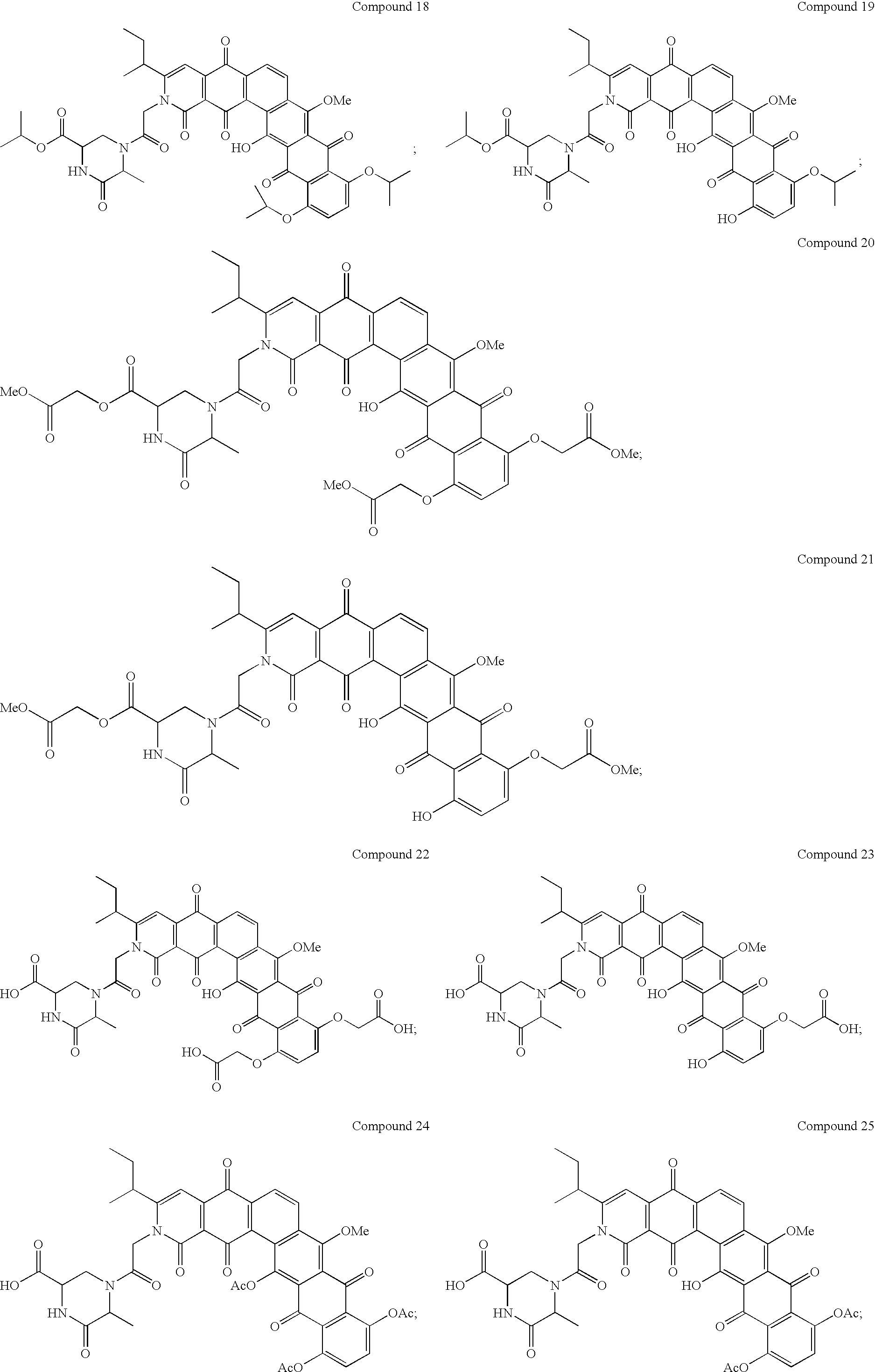 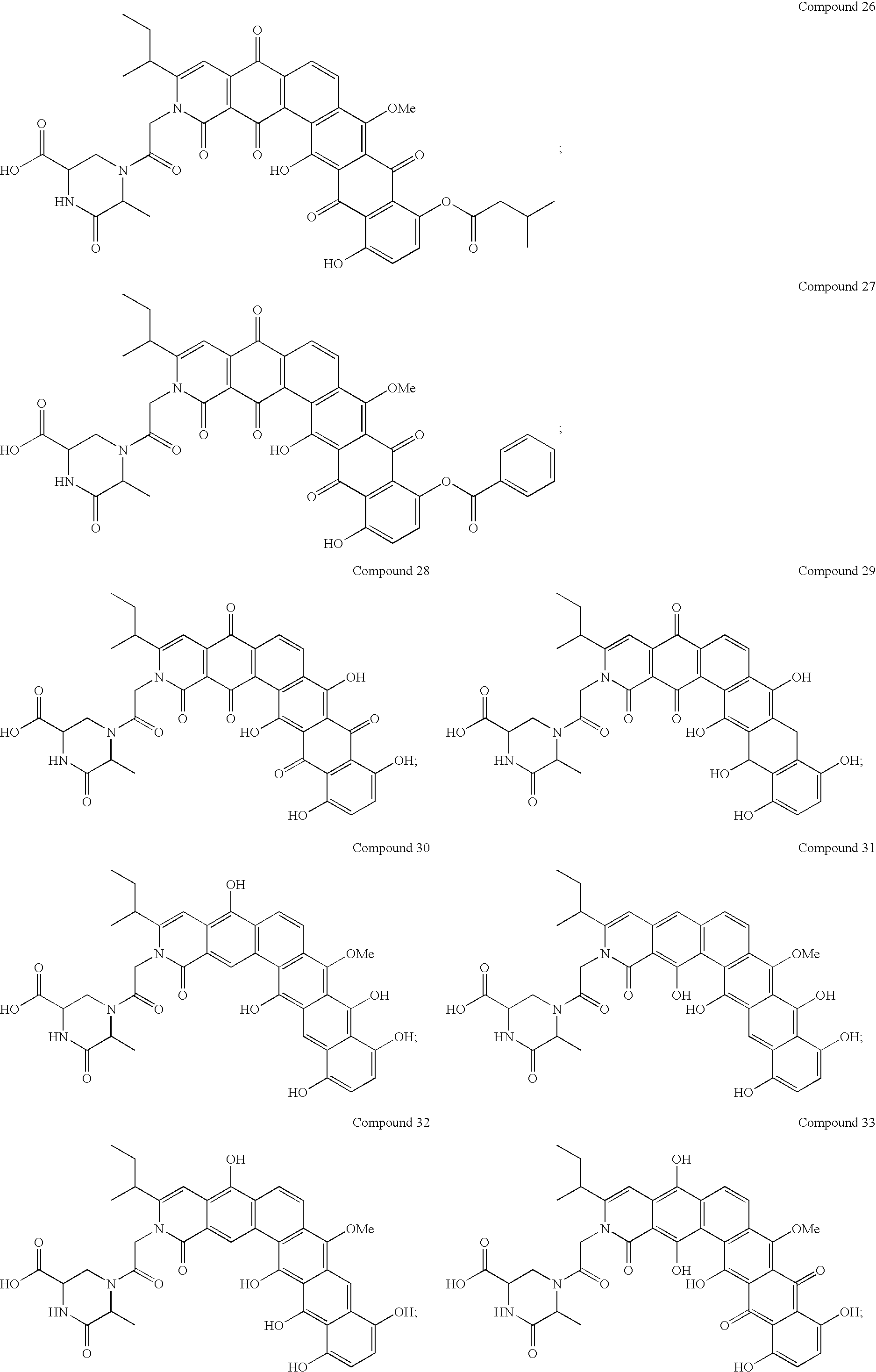 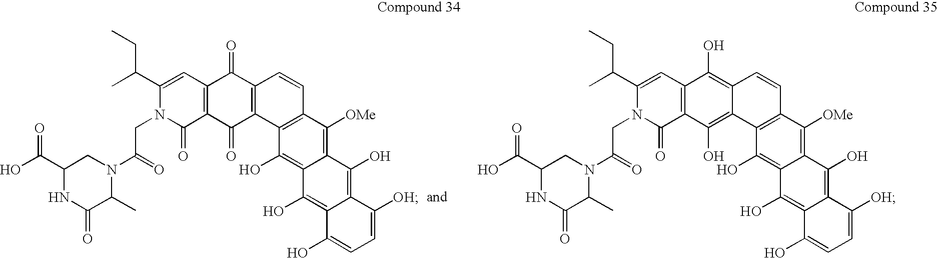
or a tautomer, an ether, ester or amide, or a pharmaceutically acceptable salt, solvate or prodrug of any one of Compounds 1-35. Certain embodiments may exclude one or more of the compounds of Formula I or Formula II.

Prodrugs of the compounds of Formula I or Formula II include compounds wherein one or more of the hydroxyl or carboxylic acid groups on the molecule is bonded to any group that, when administered to a mammalian subject, is cleaved to form the free hydroxyl or carboxylic acid group. Examples of prodrugs of hydroxyls include, but are not limited to, acetate, formate, hemisuccinate, benzoate, dimethylaminoacetate and phosphoryloxycarbonyl derivatives of hydroxyl functional groups; dimethylglycine esters, aminoalkylbenzyl esters, aminoalkyl esters or carboxyalkyl esters of hydroxy functional groups. Carbamate and carbonate derivatives of the hydroxyl groups are also included. Derivatizations of hydroxyl groups also encompassed, are (acyloxy)methyl and (acyloxy)ethyl ethers, wherein the acyl group is an alkyl group optionally substituted with groups including, but not limited to, ether, amino and carboxylic acid functionalities, or where the acyl group is an amino acid ester. Also included are phosphate and phosphonate esters, sulfate esters, sulfonate esters, which are in alkylated (such as bis-pivaloyloxymethyl (POM) phosphate triester) or in the salt form (such as sodium phosphate ester (—P(O)O⁻₂Na⁺₂)). For further examples of prodrugs, see Bundgaard et al (1992), Advanced Drug Delivery Review, vol 8, 1-38. Prodrugs of the carboxylic acids include esters and equivalents. The prodrug may also be prepared as its pharmaceutically acceptable salt.

The compounds of this invention may be formulated into pharmaceutical compositions comprised of a compound of Formula I or Formula II, in combination with a pharmaceutically acceptable carrier, as discussed in Section IV below.

### III. METHODS OF PRODUCING THE COMPOUNDS OF THE INVENTION

A. Methods of Producing the Compounds by Fermentation

In one embodiment, Compounds 1 and 2 are obtained by cultivating a

Micromonospora strain, namely Micromonospora echinospora challisensis NRRL 12255. However, it is desired and intended that the present invention is not limited to use of the particular strain. Rather, the present invention contemplates the use of other organisms producing any one of Compounds 1 or 2. Mutants or variants of Micromonospora echinospora ssp. challisensis NRRL 12255 can be naturally-occuring mutants of this organism obtained by selection, or mutants derived from this organism by known means such as X-ray irradiation, ultraviolet irradiation, treatment with a chemical mutagen such as a nitrogen mustard, N′-methyl-N′-nitro-N-nitrosoguanidine, actinophage and phage exposure, antibiotic resistance selection and the like. It is also desired and intended to include inter- and intraspecific genetic recombinants produced by genetic techniques known to those skilled in the art such as, for example, conjugation, transduction and genetic engineering techniques.

General methods for the production of mutants are easily found, for example, in the Manual of Industrial Microbiology and Biotechnology, Demain and Solomon, American Society for Microbiology, Washington D.C., 1986, which is incorporated herein by reference in its entirety. Other examples of methods are found for example, in Hesketh et al. (1997), J. Antibiotics, vol 50, no 6, 532-535, and Hosoya et al. (1998), Antimicrobial Agents and Chemotherapy, vol 42, no 8, 2041-2047, both incorporated herein by reference.

The polycyclic aromatic compounds of the invention may be biosynthesized by various microorganisms. Microorganisms that may synthesize the polycyclic aromatics of the invention include but are not limited to bacteria of the order Actinomycetales, also referred to as actinomycetes. Non-limiting examples of members belonging to the genera of Actinomycetes include Nocardia, Geodermatophilus, Actinoplanes, Micromonospora, Nocardioides, Saccharothrix, Amycolatopsis, Kutzneria, Saccharomonospora, Saccharopolyspora, Kitasatospora, Streptomyces, Microbispora, Streptosporangium, and Actinomadura. The taxonomy of actinomycetes is complex and reference is made to Goodfellow, Suprageneric Classification of Actinomycetes (1989); Bergey's Manual of Systematic Bacteriology, Vol. 4 (Williams and Wilkins, Baltimore, pp. 2322-2339); and to Embley and Stackebrandt, “The molecular phylogeny and systematics of the actinomycetes,” Annu. Rev. Microbiol. (1994) 48:257-289, each is hereby incorporated by reference in its entirety, for genera that may synthesize the compounds of the invention.

“Polycyclic aromatic-producing organism or strain” or “Compound 1/Compound 2-producing organism or strain” refers to strains of Actinomycetes, preferably of the Micromonospora genus, more preferably a species of Micromonospora echinospora, most preferably Micromonospora echinospora ssp. challisensis NRRL 12255 species or a mutant or variant thereof, capable or producing a polycyclic aromatic of the invention, i.e. Compound 1, Compound 2, or a compound of Formula I or Formula II.

The following are examples of fermentation and isolation methods, which are not to be construed as limiting the scope of the invention.

General Fermentation Methods

An actinomycete strain is selected and cultivated in culture medium containing known nutritional sources for actinomycetes, such media having assimilable sources of carbon, nitrogen, plus optional inorganic salts and other known growth factors at a pH of about 6 to about 9. Suitable media components include, but are not limited to, glucose, sucrose, mannitol, lactose, cane molasses, soluble starch, corn starch, corn dextrin, potato dextrin, linseed meal, corn steep solids, corn steep liquor, Distiller's Solubles™, dried yeast, yeast extract, malt extract, Pharmamedia™, glycerol, N-Z amine A, soybean powder, soybean flour, soybean meal, beef extract, meat extract, fish meal, Bacto-peptone, Bacto-tryptone, casamino acid, thiamine, L-glutamine, L-arginine, tomato paste, oatmeal, MgSO₄.7H₂O, MgSO₄, MgCl₂.6H₂O, CaCO₃, NaCl, Na acetate, KH₂PO₄, K₂HPO₄, K₂SO₄, Na₂HPO₄, FeSO₄.7H₂O, FeCl₂.4H₂O, ferric ammonium citrate, Kl, Nal, (NH₄)₂SO₄, NH₄H₂PO₄, NH₄NO₃, K₂SO₄, ZnCl₂, ZnSO₄.7H₂O, ZnSO₄, 5H₂O, MnCl₂.4H₂O, MnSO₄, CuSO₄.5H₂O, CoCl₂.2H₂O, phytic acid, casamino acid, proflo oil and morpholinopropanesulfonic acid (MOPS). Non-limiting examples of growth media are provided in Table 1 below.

TABLE 1
Examples of Fermentation Production Media

| Component | CA | LA | MY | SF | VB |
|---|---|---|---|---|---|
| pH*¹ | 7 | 7 | 7 | 7 | 7 |
| Glucose | 10 | | | 25 | 10 |
| Sucrose | | | | | 20 |
| Maltose | | | 4 | | |
| Cane molasses | 15 | | | 3.75 | 20 |
| Soluble starch | | 25 | | | |
| Potato dextrin | 40 | | | | |
| Dried yeast | | 5 | | | |
| Yeast extract | | | 4 | | |
| Malt extract | | | 10 | | |
| NZ-Amine A | 10 | | | 1.25 | |
| Soybean powder | | 15 | | 18.75 | |
| Soytone-peptone | | | | | 5 |
| MgSO₄.7H₂O | 1 | | | | |
| CaCO₃ | 2 | 2 | | 3 | 2.5 |
| Na acetate | | | | 8 | |

Unless otherwise indicated all the ingredients are in g/L.
*¹The pH is adjusted as marked prior to the addition of CaCO₃.

The culture media inoculated with a polycyclic aromatic-producing microorganism may be aerated by incubating the inoculated culture media with agitation, for example, shaking on a rotary shaker, a shaking water bath, or in a fermentor. Aeration may also be achieved by the injection of air, oxygen or an appropriate gaseous mixture to the inoculated culture media during incubation.

General Isolation Methods

Following cultivation, the polycyclic aromatic compound can be extracted and isolated from the cultivated culture media, from mycelial cake or fermentation broth, or both, by techniques known to a person skilled in the art and/or disclosed herein, including for example centrifugation, precipitation, chromatography, adsorption, filtration. The mycelial cake and fermentation broth are optionally separated by centrifugation and decantation prior to extraction.

In one example, the cultivated culture media, optionally acidified at pH 2-5, is mixed with a suitable organic solvent such as n-butanol, n-butyl acetate or 4-methyl-2-pentanone, the organic layer can be separated for example, by centrifugation followed by the removal of the solvent, by concentration or by evaporation to dryness under vacuum. The resulting residue is optionally reconstituted with for example water, diethyl ether, ethanol, ethyl acetate, methanol or a mixture thereof, and re-extracted in a two-phase system with a suitable organic solvent such as hexane, carbon tetrachloride, methylene chloride or a mixture thereof. Further purification of the compounds is accomplished by the use of standard techniques, such as chromatography, including normal and reverse phase HPLC (High performance liquid chromatography), HSCC chromatography (High speed counter current), Sephadex™ LH-20 chromatography, silica gel chromatography, filtration on resin (e.g. Diaion™ HP-20), and the like. Isolation or purification is also accomplished by precipitation, centrifugation or crystallization followed by decantation or filtration to obtain the desired compound from a residue by the use of appropriate solvents.

In another example, the whole fermentation broth, at harvest, is acidified with an appropriate acid (e.g. sulfuric acid), to a pH between 2 and 5, and further extracted with an organic solvent (e.g. ethyl acetate, diethyl ether, t-butyl methyl ether, toluene and the like), and concentrated in vacuo. The residue is optionally purified by any of the above-mentioned techniques.

B. Production of the Compounds of Formula I by Chemical Modifications

The polycyclic aromatic compound biosynthesized by microorganisms are optionally subjected to random and/or directed chemical modifications to form derivatives or structural analogs of Compounds 1 or 2 defined as the compounds of Formula I or Formula II. Polycyclic aromatics of Formula I or Formula II are generated by biofermentation followed by standard organic chemical modification of the polycyclic aromatics produced. Preferred polycyclic aromatic for chemical modification includes any one of Compounds 1 or 2. General principles of organic chemistry required for making and manipulating the compounds of Formula I or Formula II, including functional moieties, reactivity and common protocols are described, for example, in J. March, “Advanced Organic Chemistry”, 4^{th} Edition, John Wiley & Sons, New York (1992), which is incorporated herein by reference in its entirety. In addition, it will be appreciated by one of ordinary skill in the art that the synthetic methods described herein may use a variety of protecting groups, whether or not they are explicitly described. A “protecting group” as used herein means a moiety used to block one or more functional moieties such as reactive groups including oxygen, sulfur or nitrogen so that a reaction may be carried out selectively at another reactive site in a polyfunctional compound. General principles for the use of protective groups, their applicability to specific functional groups and their uses are described for example in T. H. Greene and P. G. M. Wuts, Protective Groups in Organic Synthesis, 3^{rd} Edition, John Wiley & Sons, New York (1999).

Alcohols are protected with, for example: silyl ethers (TMS: trimethylsilyl, TIPS: triisopropylsilyl), acetals (MOM: methyloxymethyl, BOM: benzyloxymethyl), esters (acetate, benzoyl) and ethers (Bn: benzyl). Alcohols are deprotected by conditions such as: TBAF (tetrabutylammonium fluoride) for silyl ethers, aqueous acid catalysis for acetals and esters, saponification for esters, and hydrogenolysis for Bn and BOM. Carboxylic acids are protected with, for example, esters such as methyl, ethyl, t-butyl or benzyl ester. Carboxylic acids are deprotected by conditions such as: aqueous acid hydrolysis, saponification, or hydrogenolysis in the case of the benzyl ester.

Esterification, alkylation and amide formation modification steps shown below, may require additional chemical steps if, for example, the added group has to be further modified, hydrolyzed, or deprotected, by art recognized protocols.

Those skilled in the art will readily appreciate that many synthetic chemical processes may be used to produce polycyclic aromatics based on Compound 1 or Compound 2. The following schemes are exemplary of the routine chemical modifications that may be used to produce compounds of Formula I or Formula II. Any chemical synthetic process known to a person skilled in the art providing the structures described herein may be used and are therefore comprised in the present invention. 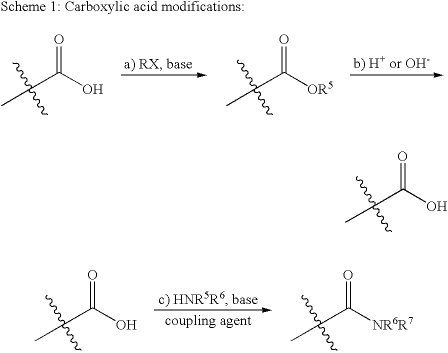
wherein, R⁵, R⁶ and R⁷ are as previously described.

In Scheme 1, carboxylic acid at position 24 (30-C(O) and 31-OH) is used for the production of esters and amides, see Example 3 for atom numbering used herein.

In Scheme 1(a), the acid moiety is esterified in a) by conditions such as alkylation, esterification or coupling conditions. Alkylation conditions include use of an alkylating agent (RX, wherein X is a leaving group like halide, diazo, mesylate or other sulfonates) and a base such as pyridine or triethylamine. Esterification conditions include use of an alcohol (ROH) with acid catalysis at high temperature. Coupling conditions include the transformation of the carboxylic acid (C(O)OH) to an activated carboxylic acid (C(O)X) such as acyl chloride (by oxalyl chloride) or to an active species produced by activation by a coupling agent (for example: EDC (1-(3-dimethylaminopropyl)-3-diisopropylethylcarbodiimide hydrochloride) and HATU (O-(7-Azabenzotriazol-1-yl)-N,N,N′,N′tetramethyluronium hexafluorophosphate) in the presence of a base, such as DIPEA (N,N-diisopropylethylamine), and the appropriate alcohol (RX is ROH). Reagents such as DMAP (4-(dimethylamino) pyridine)) or HOBT (1-hydroxybenzotriazole hydrate) may also be added to the reaction mixture as catalyst. Scheme 1(a) is used to obtain Compounds 2 to 8 from Compound 1.

In Scheme 1(b), an ester is hydrolyzed to produce the corresponding carboxylic acid in aqueous acidic or basic conditions as generally known to the art. Scheme 1(b) is used to produce Compounds 1, 15, 16, 22 and 23 respectively from Compounds 2, 14, 17, 20 and 21.

In Scheme 1(c), an amide is formed from the coupling of the carboxylic acid with an appropriate amine (HNR⁵R⁶). Coupling conditions include activating the carboxylic acid using a coupling agent (such as in a) and reacting with the amine in the presence of a base like DIPEA. Reagents such as DMAP or HOBT may also be added. The amine is selected from ammonia, or a precursor, a primary or a secondary amine. If the amine is, for example; an O-protected amino acid (for examples t-butyl ester of amino acid), then the coupling reaction is optionally followed by deprotection. Scheme 1(c) is used to obtain Compounds 9, 10 and 12 from Compound 1, and Compound 11 from Compound 24. 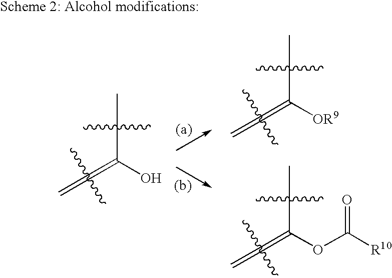
wherein, R⁹ and R¹⁰ are as previously described.

In Scheme 2, alcohols at positions 4 (32-OH), 6 (33-OH) and 9 (34-OH), position 11 (35-OH when previously demethylated, as in Scheme 3), and positions 3, 5, 10, 11 and 14 (when the quinones are reduced according to Scheme 4) are independently used for the production of esters (b) and ethers (a), see Example 3 for atom numbering used herein.

In Scheme 2(a), an alcohol is etherified by an alkylating agent (R′X, wherein X is a leaving group like halide, mesylate or other sulfonates) and a base such as pyridine, triethylamine or DIPEA (N,N-diisopropylethylamine). R′X may also be a diazoalkane. Fully alkylated products, both etherified and esterified (as in Scheme 1(a) are also obtained. Scheme 2(a) is used to obtain Compounds 13, 14 and 17 from either Compound 1 or 2, Compounds 15 and 16 from Compound 1, Compounds 18 and 19 from either Compound 1 or 4, and Compounds 20 and 21 from either Compound 1 or 8.

In Scheme 2(b), an alcohol is esterified by reaction with an activated carboxylic acid (R″C(O)X), such as an acyl halide or an anhydride or mixed anhydride, or the active species produced by activation of the carboxylic acid with a coupling agent (such as in Scheme 1(a)) in the presence of a base such as DIPEA. Reagents such as DMAP or HOBT may also be added to the reaction mixture as catalyst. If the activated acid is an N-protected amino acid, the coupling is optionally followed by deprotection. Scheme 2(b) is used to obtain Compounds 24 to 27 from Compound 1.

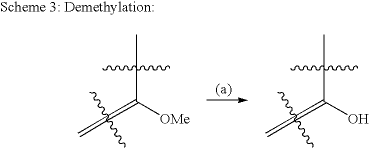

In Scheme 3, the methoxy group at position 11 (35-OMe) is demethylated according to standard procedures, such as boron tribromide, HI, HBr, thiolate ions/DMF, and LiCl in DMF under reflux (Zacharie et al. (1997), J. Chem. Soc., Perkin Trans. 1, 2925-2929). Scheme 3 is used to obtain Compound 28 from Compound 1.

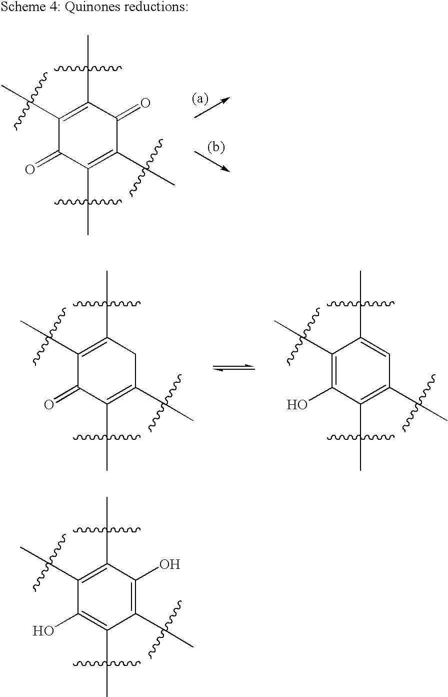

In Scheme 4(a), quinones are reduced to produce “phenol rings” using conditions such as copper and sulfuric acid or tin and hydrochloric acid (J. March, Supra, page 1210, and in Meyer et al., Org. Synth. I, 60). The cyclohexadienone intermediates produced are in equilibrium with the phenol products. Scheme 4(a) is used to produce Compounds 29 to 32 from Compound 1.

In Scheme 4(b), quinones are reduced to produce a dihydroxyaryls(hydroquinones) when treated with a reducing agent such as lithium aluminum hydride (LAH), sodium borohydride (NaBH₄), and sodium hydrosulfite (Na₂S₂O₄). Scheme 4(b) is used to produce Compounds 33 to 35 from Compound 1.

Prodrugs are prepared by routine chemical modifications such as described in Jerry March, supra, including esterification and alkylation reactions, i.e., use of activated acids or mixed anhydrides (acyl halides, use of coupling reagents, etc), and by the use of alkylating agents (R—X, wherein X is a leaving group, such as diazo, and R is the desired group). Phosphate prodrugs are prepared by phosphorylation, for example, by a procedure such as described in U.S. Pat. No. 5,561,122 (Pettit et a), in Silverberg et al. (1996), Tetrahedron Letters, vol 37, 711-774 and in Hwang and Cole (2004), Org. Lett., vol 6, no 10, 1555-1556 ((POM)₂phosphate triester from (POM)₂phosphoryl chloride), the content of which is incorporated herein by reference in their entirety.

### IV. PHARMACEUTICAL COMPOSITIONS COMPRISING THE COMPOUNDS OF THE INVENTION

In another embodiment, the invention relates to pharmaceutical compositions comprising a polycyclic aromatic of the invention or a pharmaceutically acceptable salt or prodrug thereof, as described in the preceding section, and a pharmaceutically acceptable carrier as described below. The pharmaceutical composition comprising a compound of the invention is useful as a cytotoxic agent and for inhibiting the growth of tumor, bacterial and fungal cells. These compositions are used for the treatment of such conditions in warm-blooded animals, including mammals such as humans, or as general disinfectants. This section contains examples of pharmaceutical compositions, which are not to be construed as limiting the scope of the invention.

The pharmaceutical preparations disclosed herein are prepared in accordance with standard procedures and are administered at dosages that are selected to reduce, prevent, or eliminate tumor, bacterial and fungal growth (See, e.g., Remington's Pharmaceutical Sciences, Mack Publishing Company, Easton, Pa. and Goodman and Gilman's the Pharmaceutical Basis of Therapeutics, Pergamon Press, New York, N.Y., the contents of which are incorporated herein by reference). The compositions of the present invention can be delivered using controlled (e.g., capsules) or sustained release delivery systems (e.g., bioerodable matrices). Exemplary delayed release delivery systems for drug delivery that are suitable for administration of the compositions of the invention. (preferably of Formula I or Formula II) are described in U.S. Pat. No. 4,452,775 (issued to Kent), U.S. Pat. No. 5,039,660 (issued to Leonard), U.S. Pat. No. 3,854,480 (issued to Zaffaroni).

The compositions comprising a compound of this present invention will contain from about 0.1% to about 99.9%, about 5% to about 95%, about 10% to about 80% or about 15% to about 60% by weight of the active compound.

In therapeutic use, the compounds of the present invention, or pharmaceutically acceptable salts or prodrugs thereof, can be formulated in a conventional pharmaceutical composition appropriate for oral, sublingual, intranasal, intraocular, rectal, transdermal, mucosal, topical or parenteral administration for the therapeutic or prophylactic treatment of diseases, particularly tumor, bacterial and fungal growth. Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracerebral or intracranial, intraspinal, intracisternal, and intrathecal (spinal fluids). Any known device useful for parenteral injection or infusion of drug formulations can be used to effect such administration.

The pharmaceutically-acceptable compositions of the present invention comprise one or more compounds of the present invention in association with one or more non-toxic, pharmaceutically-acceptable carriers and/or diluents and/or adjuvants and/or excipients, collectively referred to herein as “carrier” materials, and if desired other active ingredients. Pharmaceutically acceptable carriers include, for example, solvents, vehicles or medium such as saline, buffered saline, dextrose, water, glycerol, ethanol, hydrophobic carriers, and combinations thereof. The term specifically excludes cell culture medium. Hydrophobic carriers include, for example, fat emulsions, lipids, polymer matrices, biocompatible polymers, lipospheres, vesicles, particles, and liposomes.

Excipients or additives are known to the art, pharmaceutically acceptable additives, other than the active ingredient, included in a formulation and having different purposes depending, for example on the nature of the drug, and the mode of administration. Examples of generally used excipients include, without limitation: stabilizing agents, solubilizing agents and surfactants, buffers, antioxidants and preservatives, tonicity agents, bulking agents, lubricating agents, emulsifiers, suspending or viscosity agents, inert diluents, fillers, disintegrating agents, binding agents, wetting agents, lubricating agents, antibacterials, chelating agents, sweetners, perfuming agents, flavouring agents, coloring agents, administration aids, and combinations thereof.

The compositions may contain common carriers and excipients, such as cornstarch or gelatin, lactose, sucrose, microcrystalline cellulose, kaolin, mannitol, dicalcium phosphate, sodium chloride and alginic acid. The compositions may contain crosarmellose sodium, microcrystalline cellulose, sodium starch glycolate and alginic acid.

Formulations for parenteral administration can be in the form of aqueous or non-aqueous isotonic sterile injection solutions, suspensions or fat emulsions, comprising a compound of this invention as a free acid or as a salt. The parenteral form used for injection must be fluid to the extent that easy syringability exists. These solutions or suspensions can be prepared from sterile powders or granules. The compounds can be dissolved in a carrier such as a solvent or vehicle, for example, polyethylene glycol, propylene glycol, ethanol, corn oil, benzyl alcohol, glycofurol, N,N-dimethylacetamide, N-methylpyrrolidone, glycerine, saline, dextrose, water, glycerol, hydrophobic carriers, and combinations thereof.

Excipients used in parenteral preparations also include, without limitation, stabilizing agents (e.g. carbohydrates, amino acids and polysorbates), solubilizing agents (e.g. cetrimide, sodium docusate, glyceryl monooleate, polyvinylpyrrolidone (PVP) and polyethylene glycol (PEG)) and surfactants (e.g. polysorbates, tocopherol PEG succinate, poloxamer and cremophor), buffers (e.g. acetates, citrates, phosphates, tartrates, lactates, succinates, amino acids and the like), antioxidants and preservatives (e.g. BHA, BHT, gentisic acids, vitamin E, ascorbic acid and sulfur containing agents such as sulfites, bisulfites, metabisulfites, thioglycerols, thioglycolates and the like), tonicity agents (for adjusting physiological compatibility), suspending or viscosity agents, antibacterials (e.g. thimersol, benzethonium chloride, benzalkonium chloride, phenol, cresol and chlorobutanol), chelating agents, and administration aids (e.g. local anesthetics, anti-inflammatory agents, anti-clotting agents, vaso-constrictors for prolongation and agents that increase tissue permeability), and combinations thereof.

Parenteral formulations using hydrophobic carriers include, for example, fat emulsions and formulations containing lipids, lipospheres, vesicles, particles and liposomes. Fat emulsions include in addition to the above-mentioned excipients, a lipid and an aqueous phase, and additives such as emulsifiers (e.g. phospholipids, poloxamers, polysorbates, and polyoxyethylene castor oil), and osmotic agents (e.g. sodium chloride, glycerol, sorbitol, xylitol and glucose). Liposomes include natural or derived phospholipids and optionally stabilizing agents such as cholesterol.

In another embodiment, the parenteral unit dosage form of the compound can be a ready-to-use solution of the compound or a salt thereof in a suitable carrier in sterile, hermetically sealed ampoules or in sterile pre-loaded syringes. The suitable carrier optionally comprises any of the above-mentioned excipients.

Alternatively, the unit dosage form of the compound of the present invention can be in a concentrated or powder bulk form for ex tempore reconstitution in the appropriate pharmaceutically acceptable carrier at the time of delivery. In addition the above-mentioned excipients, powder forms optionally include bulking agents (e.g. mannitol, glycine, lactose, sucrose, trehalose, dextran, hydroxyethyl starch, ficoll and gelatin), and cryo or lyoprotectants.

For intravenous (IV) use, compounds of the present invention can be dissolved or suspended in any of the commonly used intravenous fluids and administered by infusion. Intravenous fluids include, without limitation, physiological saline or Ringer's™ solution.

For intramuscular preparations, a sterile formulation of compounds of the present invention or suitable soluble salts forming the compound, can be dissolved and administered in a pharmaceutical diluent such as Water-for-Injection (WFI), physiological saline or 5% glucose. A suitable insoluble form of the compound may be prepared and administered as a suspension in an aqueous base or a pharmaceutically acceptable oil base, e.g. an ester of a long chain fatty acid such as ethyl oleate.

For oral use, solid formulations such as tablets and capsules are particularly useful. Sustained released or enterically coated preparations may also be devised. For paediatric and geriatric applications, suspension, syrups and chewable tablets are especially suitable. For oral administration, the pharmaceutical compositions are in the form of, for example, tablets, capsules, suspensions or liquid syrups or elixirs, wafers and the like. For general oral administration, excipient or additives include, but are not limited to inert diluents, fillers, disintegrating agents, binding agents, wetting agents, lubricating agents, sweetening agents, flavoring agents, coloring agents and preservatives.

The oral pharmaceutical composition is preferably made in the form of a dosage unit containing a therapeutically-effective amount of the active ingredient. Examples of such dosage units are tablets and capsules. For therapeutic purposes, the tablets and capsules which can contain, in addition to the active ingredient, conventional carriers such as: inert diluents (e.g., sodium and calcium carbonate, sodium and calcium phosphate, and lactose), binding agents (e.g., acacia gum, starch, gelatin, sucrose, polyvinylpyrrolidone (Providone), sorbitol, or tragacanth methylcellulose, sodium carboxymethylcellulose, hydroxypropyl methylcellulose, and ethylcellulose), fillers (e.g., calcium phosphate, glycine, lactose, maize-starch, sorbitol, or sucrose), lubricants (e.g., magnesium stearate or other metallic stearates, stearic acid, polyethylene glycol, waxes, oils, silica and colloical silica, silicon fluid or talc), disintegrants (e.g., potato starch, corn starch and alginic acid), flavouring, coloring agents, or acceptable wetting agents. Carriers may also include coating excipients such as glyceryl monostearate or glyceryl distearate, to delay absorption in the gastrointestinal tract.

Oral liquid preparations, generally in the form of aqueous or oily solutions, suspensions, emulsions, syrups or elixirs, may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

For both liquid and solid oral preparations, flavoring agents such as peppermint, oil of wintergreen, cherry, grape, fruit flavoring or the like can also be used. It may also be desirable to add a coloring agent to make the dosage form more aesthetic in appearance or to help identify the product comprising a compound of the present invention.

For topical use the compounds of present invention can also be prepared in suitable forms to be applied to the skin, or mucus membranes of the nose and throat, and can take the form of creams, ointments, liquid sprays or inhalants, lozenges, or throat paints. Such topical formulations further can include chemical compounds such as dimethylsulfoxide (DMSO) to facilitate surface penetration of the active ingredient.

For application to the eyes or ears, the compounds of the present invention can be presented in liquid or semi-liquid form formulated in hydrophobic or hydrophilic bases as ointments, creams, lotions, paints or powders.

For rectal administration the compounds of the present invention can be administered in the form of suppositories admixed with conventional carriers such as cocoa butter, wax or other glyceride.

A compound according to this invention may also be administered in the diet or feed of a patient or animal. The diet for animals can be normal foodstuffs to which the compound can be added or it can be added to a premix.

The amount of the compound of the present invention in a unit dosage comprises a therapeutically-effective amount of at least one active compound of the present invention which may vary depending on the recipient subject, route and frequency of administration. A recipient subject refers to a plant, a cell culture or an animal such as an ovine or a mammal including a human.

According to this aspect of the present invention, the novel compositions disclosed herein are placed in a pharmaceutically acceptable carrier and are delivered to a recipient subject (including a human subject) in accordance with known methods of drug delivery. In general, the methods of the invention for delivering the compositions of the invention in vivo utilize art-recognized protocols for delivering the agent with the only substantial procedural modification being the substitution of the compounds of the present invention for the drugs in the art-recognized protocols.

Likewise, the methods for using the claimed composition for treating cells in culture utilize art-recognized protocols for treating cell cultures with cytotoxic agent(s) with the only substantial procedural modification being the substitution of the compounds of the present invention for the agents used in the art-recognized protocols.

The compounds of the present invention provide a method for treating bacterial, fungal and tumor growth and pre-cancerous or cancerous conditions. As used herein the term unit dosage refers to a quantity of a therapeutically-effective amount of a compound of the present invention that elicits a desired therapeutic response. As used herein the phrase “therapeutically-effective amount” means an amount of a compound of the present invention sufficient to prevent the onset, alleviate the symptoms, or stop the progression of a bacterial or fungal infection or pre-cancerous or cancerous condition, without undue adverse side effects (e.g. toxicity) commensurate with a reasonable benefit/risk ratio. The term “treating” is defined as administering, to a subject, a therapeutically-effective amount of at least one compound of the present invention, both to prevent the occurrence of a bacterial or fungal infection or pre-cancer or cancer condition, or to control or eliminate a bacterial or fungal infection or pre-cancer or cancer condition. The term “desired therapeutic response” refers to treating a recipient subject with a compound of the present invention such that a bacterial or fungal infection or pre-cancer or cancer condition is reversed, arrested or prevented in a recipient subject.

The compounds of the present invention can be administered as a single daily dose or in multiple doses per day. The treatment regime may require administration over extended periods of time, e.g., for several days or for from two to four weeks. The amount per administered dose or the total amount administered will depend on such factors as the nature and severity of the infection or disease state, the age and general health of the recipient subject, the tolerance of the recipient subject to the compound and the type of cancer, infection or disease.

The compounds of the present invention may be taken in combination, together or separately with any known clinically approved anti-bacterial, anti-fungal or anti-cancer to treat a recipient subject in need of such treatment.

Since the compounds of this invention possess antibacterial properties, formulations comprising same may be used for therapeutic and non-therapeutic purposes in antiseptic and disinfectant formulations. One or more compound of this invention can be formulated as, for example, a foaming detergent or solution such as a soap, shampoo, shower gel or shaving cream, as a microemulsion or micellar solution, as a spray or in simple alcoholic solutions, creams or emulsions.

Foaming detergents or solutions can include one or more conventional additives, for example, surfactants (e.g. amphoteric, anionic, cationic or non-ionic), humectants (e.g. glycols and polyethylene glycols), ethylene oxide and polypropylene copolymers, an alcohol (e.g. ethanol, isopropanol, benzyl alcohol) or a polyols (e.g. glycerol), complexing agents (e.g. for complexing Ca²⁺, Mg²⁺, and heavy metal ions), salts and buffers, natural, cellulosic or synthetic polymers (e.g. polyvinylpyrrolidone), thickening and fatting agents (e.g. polyethylene glycol distearate or copra monoethanolamide or diethanolamide), fragrance, preservatives and colorants.

Microemulsions, micellar solutions or any ternary or quaternary formulations having water/active ingredient/surfactant/co-surfactant which allows solubilization of one or more compound of this invention in water. These solutions can be used diluted or non-diluted in the form of a vasopump or propellant. Sprays are made using simple aqueous or non-aqueous solutions and may be used, for example, for making antiseptics for postoperative treatments, for the treatment of infections, burns, eczema, gluteal erythema, wounds, or acne, or for deodorants. Alcoholic solutions (e.g. 20% to 80% alcohol w/w) may be used as skin antiseptics and may contain excipients such as Azone (Nelson Research) and Transcutol (Gattefosse), which are used to help the active ingredient penetrate the keratinized layers of skin and superficial bodygrowth. Creams and emulsions comprise, in addition to one or more compound of the invention, excipients conventionally found in such preparations.

Another non-therapeutic use of the compounds of this invention is in the preparation of surface disinfectants, especially for use in the medical or veterinary sectors. These surface disinfectants may be in the form of aqueous or non-aqueous foaming detergent, sprays or nebulizers. These formulations may contain the same ingredients as above, and certain organic solvents may be added.

### V. METHOD OF INHIBITING BACTERIAL GROWTH

As described herein, the compounds of this invention possess antibacterial activity. The compounds of this invention can thus be used as antibacterial agents, for the suppression of bacterial infections, as topical antibacterial agents or as general disinfectants. Any of the pharmaceutical compositions described in the above Section V, are used in the applications described in this section.

The present invention further relates to a method for treating bacterial infection in a mammalian subject in need thereof, comprising the step of administering to the mammal a therapeutically effective amount of a polycyclic aromatic of Formula I or Formula II, a compound as described herein, or a pharmaceutically acceptable salt or prodrug thereof. The present invention relates to the use of a polycyclic aromatic of Formula I or Formula II, a compound as described herein, or a pharmaceutically acceptable salt or prodrug thereof, as a pharmaceutical for treating bacterial infection in a recipient subject in need thereof. A recipient subject refers to a warm-blooded animal such as an ovine or another mammal, including a human.

According to another embodiment, the invention provides a method of decreasing bacterial quantity in a biological sample. This method comprises the step of contacting the biological sample with a polycyclic aromatic of Formula I or Formula II, a compound as described herein, or a pharmaceutically acceptable salt or prodrug thereof. This method is effective if the number of bacteria decreases by at least 10%, and preferably more, e.g., 25%, 50%, 75% or even 100% after contacting the biological sample with a polycyclic aromatic of Formula I, a compound as described herein, or a pharmaceutically acceptable salt or prodrug thereof.

These pharmaceutical compositions effective to treat or prevent a bacterial infection, which comprise any one of Compound 1, Compound 2, a compound of Formula I as described herein, or a pharmaceutically acceptable salt or prodrug thereof in an amount sufficient to measurably decrease bacterial quantity, and a pharmaceutically acceptable carrier, are another embodiment of the present invention. The term “measurably decrease bacterial quantity”, as used herein means a measurable change in the number of bacteria between a sample containing the inhibitor and a sample not containing the inhibitor.

Agents which increase the susceptibility of bacterial organisms to antibiotics are known. For example, U.S. Pat. No. 5,523,288, U.S. Pat. No. 5,783,561 and U.S. Pat. No. 6,140,306 describe methods of using bactericidal/permeability-increasing protein (BPI) for increasing antibiotic susceptibility of gram-positive and gram-negative bacteria. Agents that increase the permeability of the outer membrane of bacterial organisms have been described by Vaara, M. in Microbiological Reviews (1992) pp. 395-411, and the sensitization of gram-negative bacteria has been described by Tsubery, H., et al, in J. Med. Chem . (2000) pp. 3085-3092.

For the method of the invention related to treatment of subjects with a bacterial infection, a typical effective unit dose of a polycyclic aromatic as described herein or a pharmaceutically acceptable salt or prodrug thereof given orally or parenterally would be from about 0.5 to about 100 mg/kg of body weight of the subject with a daily dose ranging from about 1.5 to about 300 mg/kg of body weight of the subject. A typical daily dose for an adult human is from about 50 mg to about 1.0 g.

Another preferred embodiment of this invention relates to a method, as described above, of treating a bacterial infection in a mammal in need thereof, but further comprising the step of administering to the mammal an agent which increases the susceptibility of bacterial organisms to antibiotics.

According to another preferred embodiment, the invention provides a method, as described above, of decreasing bacterial quantity in a biological sample, but further comprising the step of contacting the biological sample with an agent which increases the susceptibility of bacterial organisms to antibiotics.

Methods of decreasing bacterial quantity are effective if the number of bacteria decreases at least 10%, and preferably more, e.g., 25%, 50%, 75% or even 100% after contacting the biological sample polycyclic aromatic as described herein, or a pharmaceutically acceptable derivative, salt or prodrug thereof.

The pharmaceutical compositions and methods of this invention will be useful generally for controlling bacterial infections in vivo. Examples of bacterial organisms that may be controlled by the compositions and methods of this invention include, but are not limited to the following organisms: Streptococcus pneumoniae, Streptococcus pyogenes, Enterococcus faecalis, Enterococcus faecium, Klebsiella pneumoniae, Enterobacter spp.,

Proteus spp., Pseudomonas aeruginosa, Escherichia coli, Serratia marcesens, Staphylococcus aureus, Haemophilus influenzae, Bacillus anthracis, Mycoplasma pneumoniae, and Coagulase negative Staphylococcus including Staphylococcus epidermidis. The compositions and methods will therefore be useful for controlling, treating or reducing the advancement, severity or effects of nosocomial or non-nosocomial infections. Examples of nosocomial uses include, but are not limited to, urinary tract infections, pneumonia, surgical wound infections, bacteremia and therapy for febrile neutropenic patients. Examples of non-nosocomial uses include but are not limited to urinary tract infections, pneumonia, prostatitis, skin and soft tissue infections and intra-abdominal infections.

Since the compounds of this invention possess antibacterial properties, formulations comprising same may be used for therapeutic and non-therapeutic purposes in antiseptic and disinfectant formulations. One or more compound of this invention can be formulated as, for example, a foaming detergent, as a microemulsion or micellar solution, as a spray or in simple alcoholic solutions, in creams or emulsions.

Foaming detergents and solutions are used by nursing staff and surgeons for washing their hands, or used for cleaning dermatological lesions such as impetigo, pityriasis, and leg ulcers. Foaming detergents are used to prepare soaps, shampoos (e.g. antidandruff), shower gels or shaving creams.

Sprays and solutions are used as antiseptics for postoperative treatments, for the treatment of infections, burns, eczema, gluteal erythema, wounds, or acne, or for deodorants. The compounds are also used in combination with agents that help active ingredients to penetrate the keratinized layers of skin and superficial body growths. Such agents are, for example, Azone (Nelson Research) and Transcutol (Gattefosse). Antiseptic solutions containing such agents are used, for example, on skin before puncture, for the preparation of operative field, as hand antiseptic by nursing staff, or for treating closed infected dermatosis, folliculitis, perionychia or acne.

The compounds of this invention are also used as surface disinfectants, especially for use in the medical or veterinary sectors. These surface disinfectants may be in the form of aqueous or non-aqueous foaming detergent, sprays or nebulizers.

Treatment of bacterial infection in a subject, including mammals and humans, may be accomplished by administering a compound of the invention as a single agent, or in combination with other known antibacterial agents. Antibacterial families include, for example, antibiotics (e.g. aminoglycosides, amphenicols, ansamycins, β-lactams, lincosamides, macrolides, polypeptides, tetracyclines, and the like), and synthetic antibacterials (e.g. 2,4-diaminopyrimidines, nitrofurans, quinolones and analogs, sulfonamides, sulfones, and the like), optionally within liposomal formulations. (For more examples, see: The Merck Index, 12^{th} edition (1996), Therapeutic Category and Biological Activity Index, lists under “Antibacterial” sections; or the same sections of the more recent version: The Merck Index, 13^{th} edition (2001)).

Other examples of suitable therapeutic agents that may be used in combination with the compounds of this invention include, without limitation, penicillins and other beta lactamase inhibitors (e.g. carbapenems, cephalosporins), macrolides (including erythromycin, azithromycin, clarithromycin and ketolides), sulfonamides, aminoglycosides, quinolones (such as fluoroquinolones, e.g. ciprofloxacin), oxazolidinones, lipopeptides (such as daptomycin), tetracyclines, vancomycin, erythromycin, streptomycin, efflux pump inhibitors, lactoferrins, and cationic peptides. Such agents may be administered together with or separately from the compounds of this invention. In addition, certain patients may suffer from or may be susceptible to simultaneous infections from bacteria and one or more viruses. Those patients may benefit from simultaneous or separate co-administration of a compound or formulation according to this invention and an anti-viral agent, for example, without limitation, an anti-influenza medication such as Relenza™ (zanamivir) and Tamiflu™ (oseltamivir) or an anti-enteric virus drug such as pleconaril. Additional combination therapies may also include a compound of this invention and an anti-fungal agent, such as Cancidas™ (caspofungin acetate), Diflucan™ (fluconazole), and Mycostatin™ (nystatin). Clearly, the combination therapies described herein are merely exemplary and are not meant to limit possibilities for other combination treatments or co-administration regimens.

### VI. METHOD OF INHIBITING TUMOR GROWTH

In one aspect, the invention relates to a method for inhibiting growth and/or proliferation of cancer cells in a mammal. In another aspect, the invention provides a method for treating neoplasms in a mammal. Mammals include ungulates (e.g. sheep, goats, cows, horses, pigs), and non-ungulates, including rodents, felines, canines and primates (i.e. human and non-human primates). In a preferred embodiment, the mammal is a human.

As used herein, the terms “neoplasm”, “neoplastic disorder”, “neoplasia” “cancer,” “tumor” and “proliferative disorder” refer to cells having the capacity for autonomous growth, i.e., an abnormal state or condition characterized by rapidly proliferating cell growth which generally forms a distinct mass that shows partial or total lack of structural organization and functional coordination with normal tissue. The terms are meant to encompass hematopoietic neoplasms (e.g. lymphomas or leukemias) as well as solid neoplasms (e.g. sarcomas or carcinomas), including all types of pre-cancerous and cancerous growths, or oncogenic processes, metastatic tissues or malignantly transformed cells, tissues, or organs, irrespective of histopathologic type or stage of invasiveness. Hematopoietic neoplasms are malignant tumors affecting hematopoietic structures (structures pertaining to the formation of blood cells) and components of the immune system, including leukemias (related to leukocytes (white blood cells) and their precursors in the blood and bone marrow) arising from myeloid, lymphoid or erythroid lineages, and lymphomas (relates to lymphocytes). Solid neoplasms include sarcomas, which are malignant neoplasms that originate from connective tissues such as muscle, cartilage, blood vessels, fibrous tissue, fat or bone. Solid neoplasms also include carcinomas, which are malignant neoplasms arising from epithelial structures (including external epithelia (e.g., skin and linings of the gastrointestinal tract, lungs, and cervix), and internal epithelia that line various glands (e.g., breast, pancreas, thyroid). Examples of neoplasms that are particularly susceptible to treatment by the methods of the invention include leukemia, and hepatocellular cancers, sarcoma, vascular endothelial cancers, breast carcers, central nervous system cancers (e.g. astrocytoma, gliosarcoma, neuroblastoma, oligodendroglioma and glioblastoma), prostate cancers, lung and bronchus cancers, larynx cancers, esophagus cancers, colon cancers, colorectal cancers, gastro-intestinal cancers, melanomas, ovarian and endometrial cancer, renal and bladder cancer, liver cancer, endocrine cancer (e.g. thyroid), and pancreatic cancer.

The polycyclic aromatic is brought into contact with or introduced into a cancerous cell or tissue. In general, the methods of the invention for delivering the compositions of the invention in vivo utilize art-recognized protocols for delivering therapeutic agents with the only substantial procedural modification being the substitution of the polycyclic aromatic of the present invention for the therapeutic agent in the art-recognized protocols. The route by which the polycyclic aromatic is administered, as well as the formulation, carrier or vehicle will depend on the location as well as the type of the neoplasm. A wide variety of administration routes can be employed. The polycyclic aromatic may be administered by intravenous or intraperitoneal infusion or injection. For example, for a solid neoplasm that is accessible, the compound of the invention may be administered by injection directly into the neoplasm. For a hematopoietic neoplasm the compound may be administered intravenously or intravascularly. For neoplasms that are not easily accessible within the body, such as metastases or brain tumors, the compound may be administered in a manner such that it can be transported systemically through the body of the mammal and thereby reach the neoplasm and distant metastases for example intrathecally, intravenously or intramuscularly or orally. Alternatively, the compound can be administered directly to the tumor. The compound can also be administered subcutaneously, intraperitoneally, topically (for example for melanoma), rectally (for example colorectal neoplasm) vaginally (for example for cervical or vaginal neoplasm), nasally or by inhalation spray (for example for lung neoplasm).

The polycyclic aromatic is administered in an amount that is sufficient to inhibit the growth or proliferation of a neoplastic cell, or to treat a neoplastic disorder. The term “inhibition” refers to suppression, killing, stasis, or destruction of cancer cells. The inhibition of mammalian cancer cell growth according to this method can be monitored in several ways. Cancer cells grown in vitro can be treated with the compound and monitored for growth or death relative to the same cells cultured in the absence of the compound. A cessation of growth or a slowing of the growth rate (i.e., the doubling rate), e.g., by 50% or more at 100 micromolar, is indicative of cancer cell inhibition (see Anticancer Drug Development Guide: preclinical screening, clinical trials and approval; B. A. Teicher and P. A. Andrews, ed., 2004, Humana Press, Totowa, N.J.). Alternatively, cancer cell inhibition can be monitored by administering the compound to an animal model of the cancer of interest. Examples of experimental non-human animal cancer models are known in the art and described below and in the examples herein. A cessation of tumor growth (i.e., no further increase in size) or a reduction in tumor size (i.e., tumor volume by least a 58%) in animals treated with the compound relative to tumors in control animals not treated with the compound is indicative of significant tumor growth inhibition (see Anticancer Drug Development Guide: preclinical screening, clinical trials and approval; B. A. Teicher and P. A. Andrews, ed., 2004, Humana Press, Totowa, N.J.).

The term “treatment” refers to the application or administration of a polycyclic aromatic to a mammal, or application or administration of a polycyclic aromatic to an isolated tissue or cell line from a mammal, who has a neoplastic disorder, a symptom of a neoplastic disorder or a predisposition toward a neoplastic disorder, with the purpose to cure, heal, alleviate, relieve, alter, ameliorate, improve, or control the disorder, the symptoms of disorder, or the predisposition toward disorder. The term “treating” is defined as administering, to a mammal, an amount of a polycyclic aromatic sufficient to result in the prevention, reduction or elimination of neoplastic cells in a mammal (“therapeutically effective amount”). The therapeutically effective amount and timing of dosage will be determined on an individual basis and may be based, at least in part, on consideration of the age, body weight, sex, diet and general health of the recipient subject, on the nature and severity of the disease condition, and on previous treatments and other diseases present. Other factors also include the route and frequency of administration, the activity of the administered compound, the metabolic stability, length of action and excretion of the compound, drug combination, the tolerance of the recipient subject to the compound and the type of neoplasm or proliferative disorder. In one embodiment, a therapeutically effective amount of the compound is in the range of about 0.01 to about 750 mg/kg of body weight of the mammal. In another embodiment, the therapeutically effective amount is in the range of about 0.01 to about 500 mg/kg body weight per day. In yet another embodiment, the therapeutically effective amount is in the range of 1 to about 300 mg/kg body weight per day. The therapeutically effective doses of the above embodiments may also be expressed in milligrams per square meter (mg/m²) in the case of a human patient. Conversion factors for different mammalian species may be found in:Freireich et al, Quantitative comparison of toxicity of anticancer agents in mouse, rat, dog, monkey and man, Cancer Chemoth. Report, 1966, 50(4): 219-244, incorporated herein by reference in its entirety. When special requirements may be needed (e.g. for children patients), the therapeutically effective doses described above may be outside the ranges stated herein. Such higher or lower doses are within the scope of the present invention.

To monitor the efficacy of tumor treatment in a human, tumor size and/or tumor morphology is measured before and after initiation of the treatment, and treatment is considered effective if either the tumor size ceases further growth, or if the tumor is reduced in size, e.g., by at least 10% or more (e.g., 20%, 30%, 40%, 50%, 60%, 70%, 80%, 90% or even 100%, that is, the absence of the tumor). Prolongation of survival, time-to-disease progression, partial response and objective response rate are surrogate measures of clinical activity of the investigational agent. Tumor shrinkage is considered to be one treatment-specific response. This system is limited by the requirement that patients have visceral masses that are amenable to accurate measurement. Methods of determining the size of a tumor in vivo vary with the type of tumor, and include, for example, various imaging techniques well known to those in the medical imaging or oncology fields (MRI, CAT, PET, etc.), as well as histological techniques and flow cytometry. For certain types of cancer, evaluation of serum tumor markers are also used to evaluate response (eg prostate-specific antigen (PSA) for prostate cancer, and carcino-embryonic antigen (CEA), for colon cancer). Other methods of monitoring cancer growth include cell counts (e.g. in leukemias) in blood or relief in bone pain (e.g. prostate cancer).

The polycyclic aromatic may be administered once daily, or the compound may be administered as two, three, four, or more sub-doses at appropriate intervals throughout the day. In that case, the polycyclic aromatic contained in each sub-dose must be correspondingly smaller in order to achieve the total daily dosage. The dosage unit can also be compounded for delivery over several days, e.g., using a conventional sustained release formulation which provides sustained release of the polycyclic aromatic compound over a several day period. Sustained release formulations are well known in the art. In this embodiment, the dosage unit contains a corresponding multiple of the daily dose. The effective dose can be administered either as a single administration event (e.g., a bolus injection) or as a slow injection or infusion, e.g. over 30 minutes to about 24 hours. The compound may be administered as a treatment, for up to 30 days. Moreover, treatment of a subject with a therapeutically effective amount of a composition can include a single treatment or a series of treatments (e.g., a four-week treatment repeated 3 times, with a 2 months interval between each treatment). Estimates of effective dosages, toxicities and in vivo half-lives for the polycyclic aromatic compounds encompassed by the invention can be made using conventional methodologies or on the basis of in vivo testing using an appropriate animal model.

The polycyclic aromatic compound may be administered in conjunction with or in addition to known other anticancer treatments such as radiotherapy, or other known anticancer compounds or chemotherapeutic agents. Such agents include, but are not limited to, 5-fluorouracil, mitomycin C, methotrexate, hydroxyurea, cyclophosphamide, dacarbazine, mitoxantrone, anthracyclines (epirubicin and doxorubicin), etopside, pregnasome, platinum compounds such as carboplatin and cisplatin, taxanes such as paclitaxel and docetaxel; hormone therapies such as tamoxifen and anti-estrogens; antibodies to receptors, such as herceptin and Iressa; aromatase inhibitors, progestational agents and LHRH analogues; biological response modifiers such as IL2 and interferons; multidrug reversing agents such as the cyclosporin analogue PSC 833.

Toxicity and therapeutic efficacy of polycyclic aromatic compounds can be determined by standard pharmaceutical procedures in cell cultures or experimental animals. Therapeutic efficacy is determined in animal models as described above and in the examples herein. Toxicity studies are done to determine the lethal dose for 10% of tested animals (LD10). Animals are treated at the maximum tolerated dose (MTD): the highest dose not producing mortality or greater than 20% body weight loss. The effective dose (ED) is related to the MTD in a given tumor model to determine the therapeutic index of the compound. A therapeutic index (MTD/ED) close to 1.0 has been found to be acceptable for some chemotherapeutic drugs, a preferred therapeutic index for classical chemotherapeutic drugs is 1.25 or higher.

The data obtained from cell culture assays and animal studies can be used in formulating a range of dosage for use in humans. The dosage of compositions of the invention will generally be within a range of circulating concentrations that include the MTD. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound. Such information can be used to more accurately determine useful doses in humans. Levels in plasma may be measured, for example, by HPLC.

Animal models to determine antitumor efficacy of a compound are generally carried out in mice. Either murine tumor cells are inoculated subcutaneously into the hind flank of mice from the same species (syngeneic models) or human tumor cells are inoculated subcutaneously into the hind flank of severe combined immune deficient (SCID) mice or other immune deficient mice (nude mice) (xenograft models).

Advances in mouse genetics have generated a number of mouse models for the study of various human diseases including cancer. The MMHCC (Mouse models of Human Cancer Consortium) web page (emice.nci.nih.gov), sponsored by the National Cancer Institute, provides disease-site-specific compendium of known cancer models, and has links to the searchable Cancer Models Database (cancermodels.nci.nih.gov), as well as the NCI-MMHCC mouse repository. Mouse repositories can also be found at: The Jackson Laboratory, Charles River Laboratories, Taconic, Harlan, Mutant Mouse Regional Resource Centers (MMRRC) National Network and at the European Mouse Mutant Archive. Such models may be used for in vivo testing of polycyclic aromatic compounds, as well as for determining a therapeutically effective dose.

### VI. METHOD OF INHIBITING FUNGAL GROWTH

In one aspect, the invention relates to a method for inhibiting growth and/or proliferation of fungal cells in a subject. In another aspect, the invention provides a method for treating fungal infections in a subject, or for use as a crop protectant. The term subject refers to warm-blooded animals, and includes poultry and mammals. Mammals include ungulates (e.g. sheep, goats, cows, horses, pigs), and non-ungulates, including rodents, felines, canines and primates (i.e. human and non-human primates). In a preferred embodiment, the mammal is a human.

Fungal cells and infections that can be treated by the present method include infections caused by organisms such as species of Candida (e.g., C. glabrata, C. lusitaniae, C. parapsilosis, C. krusei, C. guilliermondii, C. tropicalis, C. pseudotropicalis), Cryptococcus neoformans, Pneumocystis carinii, Aspergillus species (e.g., A. flavus, A. fumigatus, A. nidulans), Coccidioides (e.g., Coccidioides immitis), Paracoccidioides (e.g., Paracoccidioides brasiliensis), Histoplasma (e.g., Histoplasma capsulatum), Blastomyces (e.g., Blastomyces dermatitidis), or Saccharomyces (e.g., Saccharomyces cerevisiae). Other organisms include species of Trichophyton, Microsporum or Epidermophyton (e.g., Trichophyton mentographytes, Trichophyton rubrum, Microsporum canis or Epidermophyton floccosum), or in mucosal infections caused by Candida albicans. Other organisms include species of filamentous fungi such as Geotrichum (e.g., Geotrichum clavatum), Trichosporon (e.g., Trichosporon beigelil), Blastoschizomyces (e.g., Blastoschizomyces capitatus), Sporothrix (e.g., Sporothrix schenckii), Scedosporium (e.g., Scedosporium apiosperum), Cladosporium (e.g., Cladosporium carrionii) and Pityrosporum ovale.

In view of their antifungal activity, compounds of Formula I or Formula II are useful for the treatment and/or prevention of fungal infections in human beings and animals. Such infections include superficial, cutaneous, subcutaneous and systemic mycotic infections such as respiratory tract infections, gastrointestinal tract infections, cardiovascular infections, urinary tract infections, CNS infections, candidiasis and chronic mucocandidiasis (e.g. thrush and vaginal candidiasis) and skin infections caused by fungi, cutaneous and mucocutaneous candidiasis, dermatophytoses including ringworm and tinea infections, athletes foot, paronychia, pityriasis versicolor, erythrasma, intertrigo, fungal diaper rash, candida vulvitis, candida balanitis and otitis externa. They may also be used as prophylactic agents to prevent systemic and topical fungal infections. Use as prophylactic agents may, for example, be appropriate as part of a selective gut decontamination regimen in the prevention of infection in immuno-compromised patients (e.g. AIDS patients, patients receiving cancer therapy or transplant patients). Prevention of fungal overgrowth during antibiotic treatment may also be desirable in some disease syndromes or iatrogenic states.

The compounds of Formula I or Formula II are also useful as crop antifungal agents. Species include, without limitation, phytopathogenic fungi, in particular those from the class consisting of: Deuteromycetes (e.g. Botrytis spp., Septoria spp., Pyricularia spp., Stagnospora spp., Helminthosporium spp., Fusarium spp., Cercospora spp., Rhynchosporium, spp. Pseudocercosporella, spp. and Alternaria spp.); Basidiomycetes (e.g. Puccinia spp., Rhizoctonia spp., and Hemileia); Ascomycetes (e.g. Venturia spp., Podospharera spp., Erysiphe spp., Monilinia spp. and Uncinula spp.); and Oomycetes (e.g. Phytophthora spp., Pemospora spp., Bremia spp., Pythium spp., and Plasmopara spp.). These compounds have fungicidal properties, and can be used to inhibit or to destroy the microorganisms occurring on plants or on parts of plants (the fruit, blossom, leaves, stalks, tubers or roots) of different crops of useful plants. They can also be used as dressings in the treatment of plant propagation material, especially seed (fruit, tubers, grain) and plant cuttings (for example rice), to provide protection against fungal infections and against phytopathogenic fungi occurring in the soil.

The polycyclic aromatic is brought into contact with fungal cells or tissue infected with fugal cells. In general, the methods of the invention for delivering the compositions of the invention in vivo utilize art-recognized protocols for delivering therapeutic agents with the only substantial procedural modification being the substitution of the compound of the present invention for the therapeutic agent in the art-recognized protocols. The route by which the compound is administered, as well as the formulation, carrier or vehicle will depend on the location as well as the type of fungal infection. A wide variety of administration routes can be employed. The compound may be administered systemically, for example orally, or by intramuscular, intrathecal, intravascular, intravenous, or intraperitoneal infusion or injection (for example, for systemic infections). The compound can also be administered subcutaneously, topically (for example, for skin infections), rectally, vaginally (for example for vaginal candidiasis), nasally or by inhalation spray.

The compound is administered in an amount that is sufficient to inhibit the growth or proliferation of a fungal cell, or to treat a fugal infection. The term “inhibition” refers to suppression, killing, stasis, or destruction of fungal cells. The inhibition of fungal cell growth according to this method can be monitored in several ways. Fungal cells grown in vitro can be treated with the compound and monitored for growth or death relative to the same cells cultured in the absence of the compound. A cessation of growth or a slowing of the growth rate, e.g., by 50% or more, is indicative of fungal cell inhibition. Alternatively, fungal cell inhibition can be monitored by administering the compound to an animal previously inoculated with a fungal species of interest. Examples of experimental non-human animal fungal models are known in the art. For example, in vivo evaluation of compounds of formula I can be carried out at a series of dose levels by administration (e.g. subcutaneously, orally, intraperitoneally or intravenously) to mice inoculated intravenously with a strain of Candida spp. The kidneys of the test animals may be removed and quantitated for viable Candida spp. and the reduction in infection may be determined relative to untreated control animals.

The term “treatment” when associated with the fungal treatment methods refers to the application or administration of a compound of the invention to a mammal who has a fungal infection, a symptom of a fungal infection or a predisposition toward a fungal infection, with the purpose to cure, heal, alleviate, relieve, alter, ameliorate, improve, or control the disorder, the symptoms of disorder, or the predisposition toward disorder, or application or administration of the compound to an isolated fungal cell line. The term “treating” is defined as administering, to a mammal, an amount of a polycyclic aromatic sufficient to result in the prevention, reduction or elimination of fungal cells in a mammal (“therapeutically effective amount”). The therapeutically effective amount and timing of dosage will be determined on an individual basis and may be based, at least in part, on consideration of the age, body weight, sex, diet and general health of the recipient subject, on the nature and severity of the disease condition, and on previous treatments and other diseases present. Other factors also include the route and frequency of administration, the activity of the administered compound, the metabolic stability, length of action and excretion of the compound, drug combination, the tolerance of the recipient subject to the compound and the type of fungal infection. In one embodiment, a therapeutically effective amount of the compound is in the range of about 0.01 to about 750 mg/kg of body weight of the mammal. In another embodiment, the therapeutically effective amount is in the range of about 0.01 to about 300 mg/kg body weight per day. In yet another embodiment, the therapeutically effective amount is in the range of 10 to about 50 mg/kg body weight per day. When special requirements may be needed (e.g. for children patients), the therapeutically effective doses described above may be outside the ranges stated herein. Such higher or lower doses are within the scope of the present invention.

The compound may be administered once daily, or the compound may be administered as two, three, four, or more sub-doses at appropriate intervals throughout the day. In that case, the compound contained in each sub-dose must be correspondingly smaller in order to achieve the total daily dosage. The dosage unit can also be compounded for delivery over several days, e.g., using a conventional sustained release formulation which provides sustained release of the compound over a several day period. Sustained release formulations are well known in the art. In this embodiment, the dosage unit contains a corresponding multiple of the daily dose. The effective dose can be administered either as a single administration event (e.g., a bolus injection) or as a slow injection or infusion, e.g. over 30 minutes to about 24 hours. The compound may be administered as a treatment, for up to 30 days. Moreover, treatment of a subject with a therapeutically effective amount of a composition can include a single treatment or a series of treatments (e.g., a four-week treatment repeated 3 times, with a 2 months interval between each treatment). Estimates of effective dosages, toxicities and in vivo half-lives for the compounds encompassed by the invention can be made using conventional methodologies or on the basis of in vivo testing using an appropriate animal model.

The compound may be administered in conjunction with or in addition to one or more known antifungal agents such as polyenes (e.g., amphotericin B, nystatin, and liposomal and lipid forms thereof); azole (e.g., fluconazole, intraconazole, ketoconazole, miconazole, clotrimazole, voriconazole, ZD-08070, UK-109496, SCH 56592); purin or pyrimidine nucleotide inhibitors (e.g, 5-fluorocytosine, flucytosine); polyoxin (e.g, nikkomycin Z); a pneumocandin or echinocandin derivative (e.g., cilofungin, anidulafingin (V-echinocandin),1-[(4R,5S )-5-[(2-aminoethyl)oxy]-N²-(10,12-dimethyl-1-oxotetradecyl)-4-hydroxy-L-ornithine]-5-[(3R)-3-hydroxy-L-ornithine]pneumocandin B₀ and caspofungin (CANCIDAS™)); the elongation factor inhibitor (sordarin derivatives); or other cell wall active compound such as one or more immunomodulating agents (e.g., an interferon e.g. (IFN-), interleukin e.g. (IL-1, IL-2, IL-3 and IL-8) and colony stimulating factors, [(G)-CSF, (M)-CSF and (GM)-CSF] and defensins). The data obtained from cell culture assays and in vivo studies can be used in formulating a range of dosage for use in humans. The dosage may vary depending upon the dosage form employed and the route of administration utilized. For any compound used in the method of the invention, the therapeutically effective dose can be estimated initially from cell culture assays.

In addition to the compounds of the invention, pharmaceutically acceptable salts, solvates or prodrugs of said compounds may also be employed in compositions to treat or prevent the above-identified disorders.

### EXAMPLES

Unless otherwise noted, all reagents were purchased from Sigma Chemical Co. (St. Louis, Mo.), Aldrich.

All NMR spectra were collected in deuterated solvent on a Varian 500™ Spectrometer (¹H NMR at 500 MHz, ¹³C NMR at 125 MHz). UV and mass spectra were collected on a Waters 2690™ HPLC using a photodiode array detector (PDA, 210-400 nm) coupled to a Waters Micromass™ ZQ™ mass detector.

Unless otherwise indicated, all numbers expressing quantities of ingredients, properties such as molecular weight, reaction conditions, molar equivalents (eq), percentage of binding and/or inhibition, GI₅₀, IC₅₀ and so forth used in the specification and claims are to be understood as being modified in all instances by the term “about”. Accordingly, unless indicated to the contrary, the numerical parameters set forth in the present specification and attached claims are approximations. At the very least, and not as an attempt to limit the application of the doctrine of equivalents to the scope of the claims, each numerical parameter should at least be construed in light of the number of significant figures and by applying ordinary rounding techniques. Notwithstanding that the numerical ranges and parameters setting forth the broad scope of the invention are approximations, the numerical values set in the examples, Tables and Figures are reported as precisely as possible. Any numerical values may inherently contain certain errors resulting from variations in experiments, testing measurements, statistical analyses and such.

In the following section, examples describe in detail the production of representative compounds of the present invention. The procedures are illustrations, and the invention should not be construed as being limited by the conditions they express. No attempt has been made to optimize the yields obtained in these conditions, and it would be obvious to one skilled in the art that variations in reaction times, temperature, solvent and/or reagents could increase the yields.

In addition, the materials, methods, and examples, including in vitro and in vivo efficacy, bioavailability and toxicity properties are illustrative only and not intended to be limiting. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Example 1

### Production of Compounds 1 and 2 by Fermentation

For producing Compounds 1 and 2 the strain Micromonospora echinospora challisensis NRRL 12255 was cultivated under aerobic conditions in an aqueous nutrient medium containing assimilable sources of carbon, assimilable sources of nitrogen, inorganic salts and vitamins. Thus, for instance, preferred carbon sources were maltose, sucrose, glucose, and molasses. Preferred nitrogen sources are Soytone-peptone, yeast extract and the like. Certain media were preferred for production of Compounds 1 and 2. Representative media are provided in Table 1. This strain was preferably grown at temperatures of about 28° C. to 30° C.

A. Fermentation by Stirring

Micromonospora echinospora challisensis NRRL 12255 was maintained and sporulated on agar plates of ATCC™ culture medium 172 (glucose.(10 g), soluble starch (20 g), yeast extract (5 g), N-Z-amine A (5 g, Sigma C0626), reagent grade CaCO₃ (19), Agar (15 g), distilled water (1 L)). The innoculum for the production phase was prepared by transferring the surface growth obtained from the surface of the agar plate to a 125-mL flask containing 25 mL of FBB medium (potato dextrin (24 g), beef extract (3 g), Bacto-Casitone (5 g), glucose (5 g), yeast extract (5 g), CaCO₃ (4 g) made up to one litre with distilled water). The pH of the medium was adjusted at 7 before adding calcium carbonate and then autoclaved. The flasks were shaken (250 rpm) for about 70-72 hours at 28° C. Then, 10 mL of the inoculum culture was used to inoculate each 2-L flasks containing 500 mL of either sterile production media MY or VB. The fermentation batches were incubated aerobically under stirring (250 rpm) at 28° C. for a period of 4 days.

Compounds 1 and 2 were preferably produced using production medium MY. Compound 1 was also preferably produced in media VB. Other media for the production of Compounds 1 and 2 are presented in Table 1.

B. Fermentation in Fermentor

The innoculum was prepared by transferring the surface growth to 2-L baffled flasks containing 500 mL sterile medium FBB (as described above). After incubation at 28° C. for 70-72 hours (shaken at 250 rpm), 300 mL was taken from the inoculum culture and transferred to a production fermentor (BioFlo 110™ Fermentor, New Brunswick Scientific, Edison, N.J., USA) containing 10 L medium MY. The fermentor was set at 28° C. with a dissolved oxygen level controlled at 25% in a cascade looped with agitation that varied between 150-450 RPM, with aeration set at 0.5 V/V/M. The fermentation continued for a period of 120 h.

Other preferred media for production of Compound 1 are CA, LA, SF.

### Example 2

### Isolation and Purification of Compounds 1 and 2

Procedure 1: (for 20×500 mL of Fermentation) (Compound 1 Isolated)

The whole fermentation broth at harvest was adjusted to pH 3.5 with sulfuric acid and extracted with ethyl acetate (VN). The ethyl acetate fraction was separated and concentrated in vacuo. The residue was dissolved in methanol (12 mL) and the soluble part subjected to Sephadex™ LH-20 column (3.5×100 cm), eluting with methanol under gravity. The fractions (10 min/fraction) were collected after 2 hours of sample injection. Fractions 17 to 21 were pooled and concentrated to 10 mL to give a red precipitate, which was collected by centrifugation to give pure Compound 1 (7.93 mg).

Procedure 2: (for 12×500 mL of Fermentation) (Compound 1 Isolated)

a) The whole fermentation broth at harvest was centrifuged at 3500 rpm for 20 minutes and the supernatant liquid (A) was decanted. The residual mycelial pellet was treated with methanol (1.2 L), stirred and centrifuged. The methanolic supernatant liquid was removed and the mycelial solid was extracted with acetone, the same manner as the methanol extraction. The methanol and acetone extracts were discarded. The mycelial cake was further extracted with methanol/water (1:4,1.2 L), stirred, centrifuged and separated to give the B fraction (methanol/water).

b) The supernatant from the culture broth (A) was stirred with Diaion™ HP-20 resin (1.2 L) for 20 minutes and filtered (A1). The resin was further washed with water (1.2 L, A2), which was combined to the first filtrate (A1). The A1+A2 fraction was combined with the B fraction of a) and coated on Diaion™ HP-20 resin (2 L). The resin was washed with 2 L of each of: Methanol/Water (1:9), M/W (3:7), M/W (1:1) and 0.1% acetic acid in water. The resin was finally washed three times with 2 L of methanol/acetone (1:1) to give C fraction. The C fraction was concentrated to dryness, re-suspended in water (100 mL) and extracted with ethyl acetate to give D fraction, which was concentrated to dryness.

c) Fraction D was dissolved in DMSO (methyl sulfoxide) and purified by multiple injection on HPLC (Waters 600 Controller™, NovaPak™ C-18 25×250 mm column) using a, buffered with 0.01% trifluoroacetic acid (TFA), gradient of water and acetonitrile (MeCN) (20 mL/min, Water/MeCN 95:5 to 55:45 (0-16 min) and 55:45 to 0:100 (16-20 min)). Fractions with retention time 19.4 minutes were combined and concentrated to a residue. The residue was washed with methanol (2×2 mL) to give pure Compound 1 (5.12 mg).

Procedure 3: (for 20×500 mL of Fermentation) (Compounds 1 and 2 Isolated)

a) The whole fermentation broth at harvest was adjusted to pH 3.5 with sulfuric acid and extracted with ethyl acetate (VN). The ethyl acetate fraction was separated and concentrated in vacuo. The residue was dissolved in DMSO (2 mL) and diluted with methanol (18 mL). The suspension was centrifuged (3500 rpm for 10 min) and the supernatant subjected to Sephadex™ LH-20 column (3.5×100 cm), eluting with methanol under gravity. The fractions (10 min/fraction) were collected after 2 hours of sample injection. Fractions 2 to 10 were pooled and further purified.

b) The pooled fractions obtained from the LH-20 column were purified by multiple injection on HPLC (Waters 600 Controller™, NovaPak™ C-18 25×250 mm column) using a, buffered with 0.01% acetic acid, gradient of water and acetonitrile (MeCN) (20 mL/min, Water/MeCN 80:20 to 20:80 (0-15 min) and 20:80 to 0:100 (15-22 min)). Fractions with retention times 14.5 and 17.1 minutes were respectively collected and concentrated in vacuo to give pure Compound 1 (8.63 mg) and Compound 2 (0.5 mg).

### Example 3

### Structure Data of Compounds 1 and 2

Compounds 1 and 2 were produced by fermentation as described in Example 1 and isolated as described in Example 2. NMR spectra were collected in DMSO-d₆ ((dimethyl sulfoxide)-d₆) on a Varian 500™ Spectrometer (¹H NMR at 500 MHz, ¹³C NMR at 125 MHz) at 50° C. for Compound 1, and at room temperature for Compound 2. UV and mass spectra were collected by Waters 2690™ HPLC using a photodiode array detector (PDA, 210-400 nm) coupled to a Waters Micromass™ ZQ™ mass detector. The data presented in the Tables are referred to the arbitrary atom numbering as follows:

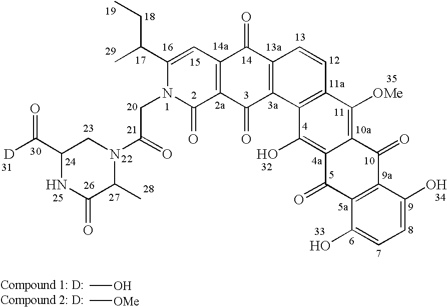

The molecular formulae and the chemical structures of Compounds 1 and 2 were established based on their mass spectra, UV, ¹H and ¹³C NMR data measured on very pure material. Molecular formulae, UV λₘₐₓ absorption (sh: shoulder) as well as the calculated molecular weights (major isotope) of Compounds 1 and 2 were identified as follows:

Compound 1:

| | |
|---|---|
| Molecular formula: | C₃₈H₃₁N₃O₁₃ |
| Calculated mass: | 737.19 g/mol |
| UV (λₘₐₓ): | 249, 280 (sh), 335 (sh), 508 nm |

Compound 2:

| | |
|---|---|
| Molecular formula: | C₃₉H₃₃N₃O₁₃ |
| Calculated mass: | 751.20 g/mol |
| UV (λₘₐₓ): | 249, 280 (sh), 335 (sh), 508 nm |

The UV spectra (by PDA) showed an absorption band at λₘₐₓ 508 nm for Compounds 1 and 2, in accordance with highly conjugated systems.

Analysis of the mass spectra of Compound 1 (detailed in Table 2) gave masses (m/z) of 738.0 and 735.9 respectively for positive ion (M+H)⁺ and negative ion (M−H)⁻ modes for a molecular ion (M) of 737.0, which confirmed a molecular formula of C₃₈H₃₁N₃O₁₃and a calculated molecular weight of the major isotope of 737.19.

Analysis of the Mass Spectra of Compound 2 (detailed in Table 2) gave masses (m/z) of 752.6 and 750.4 respectively for positive ion (M+H)⁺ and negative ion (M−H)⁻ modes for a molecular ion (M) of 751.5, which confirmed a molecular formula of C₃₉H₃₃N₃O₁₃ and a calculated molecular weight of the major isotope of 751.20.

Fragments in the mass spectra also confirmed structure assignment of Compounds 1 and 2. For example, in the positive ion mode, the breakage of the C₂₁-N₂₂ bound to lose the piperazinone moiety occurred in both Compounds 1 and 2. Compound 1 and 2 respectively lost fragments of mass 158 (C₆H,₀N₂0₃) and mass 172 (C₇H₁₂N₂O₃The residual fragments from Compound 1 gave a mass of 580.0. For Compound 2, the loss of the piperazinone group was accompanied by the loss of a methyl group to give a residual fragment of mass 580.4. This last observation further confirmed the presence of the methyl ester.

TABLE 2
Mass Spectrometry data for Compounds 1 and 2

| Compound | Ionization | Mass (m/z) | Fragment |
|---|---|---|---|
| 1 | ES+ | 760.0 | (M + Na)⁺ |
| | | 738.0 | (M + H)⁺ |
| | | 580.0 | (M + H − P¹)⁺ |
| | ES− | 735.9 | (M − H)⁻ |
| 2 | ES+ | 774.6 | (M + Na)⁺ |
| | | 752.6 | (M + H)⁺ |
| | | 580.4 | (M + H − P²)⁺ |
| | ES− | 750.4 | (M − H)⁻ |

1. P: piperazinone (C₆H₁₀N₂O₃) fragment from breaking of the C²¹—N²² bound
2. P: piperazinone-OMe (C₇H₁₂N₂O₃) fragment from breaking of the C²¹—N²² bound

Compounds 1 and 2 structure elucidation was also based on NMR data. The NMR data shown in Table 3 were based on ¹H NMR spectra, and multidimensional pulse sequences gCOSY, gDQCOSY, gHSQC, and gHMBC.

TABLE 3
¹H NMR (δ_{H}, ppm) Data of Compounds 1 and 2 in DMSO-D₆

| As- | | | |
|---|---|---|---|
| sign- | | | |
| ment | Compound 1 | Compound 2 | Group |
| 7 | 7.47 | 7.19 | CH |
| 8 | 7.44 | 7.12 | CH |
| 12 | 8.39 | 8.11 | CH |
| 13 | 8.63 | 8.23 | CH |
| 15 | 6.73 | 6.69 | CH |
| 17 | 2.67 | 2.62 | CH |
| 18 | 1.74, 1.64 | 1.74, 1.62 | CH₂ |
| 19 | 0.91 | 0.90 | CH₃ |
| 20 | 5.23 (5.12)^{a} | 5.09 | CH₂ |
| 23 | 4.40, 3.81 (4.65, 3.38)^{a} | 4.40, 3.84 (4.57, 3.42)^{a} | CH₂ |
| 24 | 4.17 (4.01)^{a} | 4.34 (4.17)^{a} | CH |
| 25 | 8.30 (8.20)^{a} | 8.51 (8.37)^{a} | NH |
| 27 | 4.67 (4.54)^{a} | 4.75 (4.53)^{a} | CH |
| 28 | 1.60 (1.32)^{a} | 1.60 (1.32)^{a} | CH₃ |
| 29 | 1.27 | 1.24 | CH₃ |
| 31 | 11.97 | 3.61 | OH (OCH₃)^{b} |
| 32 | 14.64 | N/A* | OH |
| 33 | 13.19 | 13.28 | OH |
| 34 | 13.19 | 13.28 | OH |
| 35 | 4.04 | 3.86 | CH₃ |

^{a}Data in brackets are from a second rotamer
^{b}OH in Compound 1, OCH₃ in Compound 2
*N/A: Not assigned

Knowledge of published polycyclic aromatics structures and their elucidation (for example: echinosporamicin in Haiyin He et al, Helvetica Chimica Acta, Vol. 87,1385-1391 (2004)) was helpful in the structure determination of Compounds 1 and 2. The low number of proton signals was easily assigned as above by virtue of their multiplicity, integrations, chemical shifts and correlations (short or long range ¹H—¹H and ¹H—¹³C correlations). Two rotameric forms were also observed. The bond formed by C²¹ and N²² of Compounds 1 and 2 has a slow equilibrium between two forms having a high-energy rotation barrier. This observation further confirmed the structure of Compounds 1 and 2.

### Example 4

### In vitro Tumor Growth Inhibition of Compound 1

A. In vitro Activity of Compounds 1 and 2 Against 4 Tumor Cell Lines:

Compounds were tested in four cell lines: HT-29 (colorectal carcinoma), SF268 (CNS), MDA-MB-231 (mammary gland adenocarcinoma) and PC-3 (prostate adenocarcinoma). Procedure used for each series of tests are described below.

TABLE 4
Tumor growth inhibition activity of Compounds 1 and 2 (GI₅₀)

| Cell line | Compound 1 (μM) | Compound 2 (μM) |
|---|---|---|
| HT-29 | 8.85 | 16.1 |
| MDA-MB-231 | 14.6 | 4.04 |
| PC-3 | 11.1 | 4.61 |
| SF-268 | 3.36 | 2.63 |

In vitro cytotoxic activities (GI₅₀) of Compounds 1 and 2 shown in Table 4 were determined using propidium iodide (PI) as being the concentration of drug resulting in 50% growth inhibition, and by using the following method.

Two 96-well plates were seeded in duplicate with each cell line at the appropriate inoculation density (HT29: 3,000; SF268: 3,000; PC-3: 3,000; and MDA-MB-231: 7,500 cells) and according to the technical data sheet of each cell line (rows A-G, 75 μL of media per well). Row H was filled with medium only (150 μL, negative control-medium). The plates were incubated at appropriate temperature and CO₂ concentration for 24 hrs.

Test Compounds were prepared as 15× stock solutions in appropriate medium and corresponding to 450, 45, 0.45, 0.045, and 0.0045 μM (prepared on the day of the experiment). An aliquot of each was diluted 7.5-fold in appropriate test medium to give a set of six concentration solutions (60, 6, 0.6, 0.06, 0.006, and 0.0006 μM). A 75 μL aliquot of each concentration was added to each corresponding well (rows A to F) of the second plate. Row G was filled with 75 μL of medium/0.6% DMSO (negative control-cells). The second plate was incubated at appropriate temperature and CO₂ concentration for 96 hrs.

First Plate: Pi (30 μL, 50 μg/mL) was added to each well of the first plate without removing the culture medium. The plate was centrifuged (Sorvall Legend-RT, swinging bucket) at 3500 rpm/10 min. Fluorescence intensity (Thermo, Varioskan, λₑₓ: 530 nm; λₑₘ: 620 nm) was measured to give the first measurement, dead cells (DC at T₀; before freezing). Two round of Freeze (−80° C.)/Thaw (37° C.) were done. Fluorescence intensity was determined to give the second measure, total cells (TC at T₀; after freeze/thaw)

Second plate was processed as the first one, except there were three rounds of freeze/thaw instead of two. First measurement gave the treated dead cells value (TDC), and the second measurement gave the treated total cells value (TTC). Both values were collected for each treated well and control (CTC and CDC).

Each value (DC, TC, TDC, TTC, CTC and CDC) was corrected by removing the background value (medium only) to give the value (FU_{DC(T=0)}, FU_{TC(T=0)}, FU_{TDC}, FU_{TTC}, FU_{CTC and FU}_{CDC}) used in the calculation of the T/C (%) (Treated/Control) for each concentration. T/C (%) for each concentration is calculated using the following formula:

$$T / C ( \% ) = \frac{( {FU}_{TTC} - {FU}_{TDC} ) - ( {FU}_{TC ( T = 0 )} - {FU}_{DC ( T = 0 )} ) \times 100}{( {FU}_{CTC} - {FU}_{CDC} ) - ( {FU}_{TC ( T = 0 )} - {FU}_{DC ( T = 0 )} )}$$

The GI₅₀ value emphasizes the correction for the cell count at time zero for cell survival. The T/C values are transposed in a graph to determine GI₅₀ values, the concentration at with the T/C is 50%.

B. In vitro Cytotoxic Activity of Compound 1 in a 36 Tumor Cell Lines Panel:

Culture conditions and activity evaluations of Compound 1 against 36 cancer cell lines were done as described below. Results were expressed as the concentration of drug which inhibits 50% of the cell growth (IC₅₀, calculated using the formula: [Ti/C]×100=−50). The low micromolar to nanomolar levels of IC₅₀ values shown in Table 5 demonstrated a pharmacologically relevant cytotoxic activity of Compound 2 against a variety of 36 tumor types including melanomas, pancreatic, lung, colon, gastric, bladder, renal, CNS, head and neck, prostate, uterus, ovarian and breast carcinomas.

TABLE 5
In vitro profiles of Compound 1 (IC₅₀)

| | | | | Histology | IC₅₀ |
|---|---|---|---|---|---|
| # | Type | Cell Line | Origin | in nude mice | (μM) |
| 1 | Bladder | T24 | ATCC | transitional cell ca | 5.15 |
| 2 | Bladder | 1218L | Xenograft | urotheliall adeno ca | 18.4 |
| 3 | Colon | HCT116 | NCI | adeno ca, pd | 8.24 |
| 4 | Colon | HT29 | NCI | adeno ca, pd | 5.77 |
| 5 | CNS | 498NL | Xenograft | glioblastoma | 3.70 |
| 6 | CNS | SF268 | NCI | nd | 1.52* |
| 7 | Gastric | 251L | Xenograft | adeno ca, pd | 0.72* |
| 8 | Head & | 536L | Xenograft | hypopharynx ca | 22.5 |
| | Neck | | | | |
| 9 | Lung | 1121L | Xenograft | large cell ca | 0.63* |
| 10 | Lung | 289L | Xenograft | adeno ca | 5.04 |
| 11 | Lung | 526L | Xenograft | adeno ca | 3.09 |
| 12 | Lung | 629L | Xenograft | adeno ca | 11.9 |
| 13 | Lung | 529L | Xenograft | large cell ca, | 2.20 |
| 14 | Lung | H460 | NCI | large cell ca | 6.08 |
| 15 | Mammary | 401NL | Xenograft | pap adeno ca, wd | <0.41* |
| 16 | Mammary | MCF7 | NCI | mamma ca | 4.37 |
| 17 | Melanoma | 276L | Xenograft | mm, amelanotic | 8.26 |
| 18 | Melanoma | 394NL | Xenograft | mm, amelanotic, pd | 9.99 |
| 19 | Melanoma | 462NL | Xenograft | mm, amelanotic | 24.7 |
| 20 | Melanoma | 514L | Xenograft | mm, melanotic | 10.7 |
| 21 | Melanoma | 520L | Xenograft | mm, melanotic | 7.25 |
| 22 | Ovarian | 1619L | Xenograft | adeno ca, md | 19.6 |
| 23 | Ovarian | 899L | Xenograft | pap serous ca, md | 34.9 |
| 24 | Ovarian | OVCAR3 | NCI | adeno ca, md | 26.2 |
| 25 | Pancreas | 1657L | Xenograft | adeno ca, md | 20.0 |
| 26 | Pancreas | PANC1 | ATCC | nd | 14.4 |
| 27 | Prostate | 22RV1 | ATCC | adeno ca, md | 1.65* |
| 28 | Prostate | DU145 | NCI | adeno ca, md | <0.41* |
| 29 | Prostate | LNCAP | DSMZ | adeno ca, md | 0.72* |
| 30 | Prostate | PC3M | NCI | adeno ca, pd | 0.91* |
| 31 | Pleuramesot. | 1752L | Xenograft | pleuromesothelioma | 4.23 |
| 32 | Renal | 1781L | Xenograft | renal ca | 7.54 |
| 33 | Renal | 393NL | Xenograft | hypernephroma, wd | 9.08 |
| 34 | Renal | 486L | Xenograft | hypernephroma, pd | 20.7 |
| 35 | Renal | 944L | Xenograft | hypernephroma | 14.9 |
| 36 | Uterus | 1138L | Xenograft | carcinosarcoma, wd | <0.41* |
| | | | | Mean of all cell | 9.34 |
| | | | | lines: | |

ca = carcimoma,
pd = poorly differentiated,
pap = papillary,
md = moderately differentiated,
wd = well differentiated,
mm = malignant melanoma,
nd = not done
*IC50 less than 5 fold that of Mean IC50 of all cell lines

The compound was dissolved at 10 mM in DMSO. Dilution in vehicle to concentrations of 30, 10, 3, 1 and 0.3 μM were prepared immediately before assays. Depending on the cell line's growth characteristics, 4000-10000 cells were plated in two 96-wells pates (day 0) and incubated for 16 hours. The following day, propidium iodide was added to one of the two plates. Test compound was added to the second plate, as well as vehicle control, and cells further incubated for 96 hours. The compound was tested at each concentration and in triplicates. The equivalent cell number was determined after adding propidium iodide by measuring the signal by fluorescence (Ti for test article, C for control). IC₅₀ results were calculated using the formula above and are shown in Table 5.

### Example 5

### Pharmacokinetic Profile and Toxicity of Compound 1

A. Toxicity of Compound 1:

In vitro toxicity: Sheep red blood cells were exposed to Compound 1 for 2 hrs at 37° C. at concentrations ranging from 2 to 64 μg/mL. Hemolytic activity was determined by monitoring the amount of hemoglobin released spectrophotometrically. Compound 1 did not display hemolytic activity up to 64 μg/mL when prepared either in DMSO or in 6% Tween/4.5% Glycocholate/5% EtOH formulation.

In vivo toxicity: With CD1 mice, the maximum tolerated dose (MTD) for a single-dose via i.v. or i.p. routes in 1% SDS formulation was ≧5 mg/kg. The acute toxicity in CD-1 nu/nlu mice of Compound 1 was also determined in 6% Tween/4.5% Glycocholate/5% EtOH formulation. When given i.p., MTD was ≧50 mg/kg; MTD was 25 mg/kg when Compound 1 was administered i.v. In addition, necrosis at the injection site was observed with this route of administration.

Repeated dose toxicity was evaluated in CRL nu/nu mice (6% Tween/4.5% Glycocholate/5% EtOH formulation). The MTTD (maximum total tolerated dose) was 25 mg/kg with a qd×4 dosage regimen. Adaptive hypertrophy and hyperplasia were observed in spleen and liver following repeated dosage

B. In vivo Pharmacokinetic Profile of Compound 1:

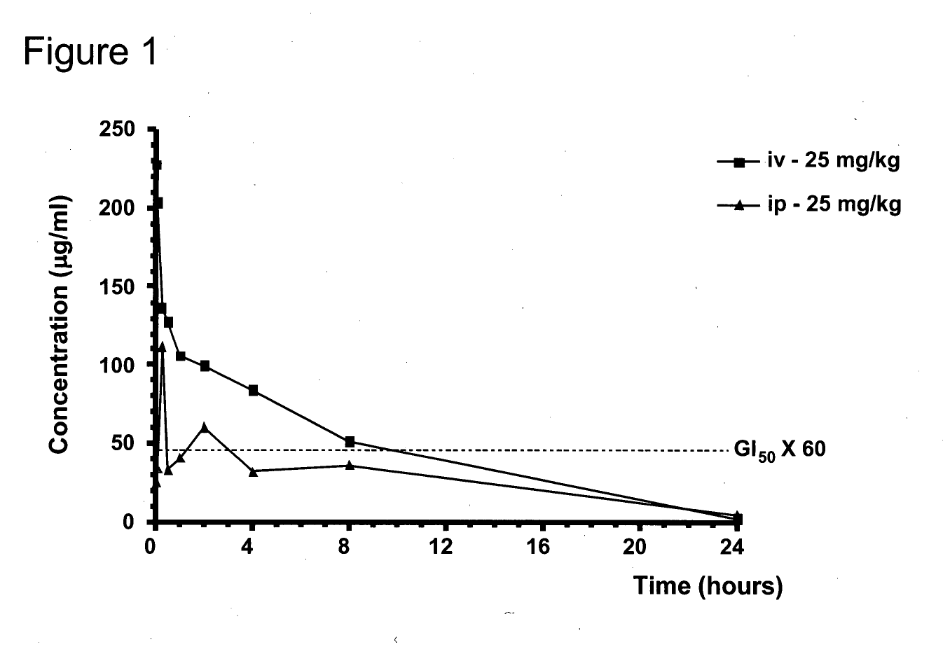

Pharmacokinetic profile of Compound 1 was determined in CD-1 mice (FIG. 1). CD1 female mice (6 weeks of age) received a single intravenous or intraperitoneal dose of Compound 1 (25 mg/kg; 5 mL/kg) in % Tween/4.5% Glycocholate/5% EtOH formulation. Blood was collected into EDTA-containing tubes by cardiac puncture. Quantification of Compound 1 plasma levels was done by high performance liquid chromatographic-mass spectrometric method. Cₘₐₓ values represent the maximum observed plasma concentration and AUC represents the area under the plasma concentration versus time curve (Table 6).

TABLE 6
In vivo pharmacokinetic profile of Compound 1

| | Cmax (μg/ml) | AUC (μg · hr/ml) |
|---|---|---|
| iv | 227 | 1175 |
| ip | 111 | 649 |

Example 6

### In vitro Antimicrobial Activity of Compounds 1 and 2

A. In vitro Antibacterial Activity of Compounds 1 and 2:

Antibacterial activity of Compounds 1 and 2 (Table 7) was measured by determining the minimal inhibitory concentration (MIC) necessary to obtain a complete inhibition of bacteria growth in seven indicator strains, namely Staphylococcus aureus (ATCC™ 6538P), Staphylococcus aureus MRS3 (ATCC™ 700699), Enterococcus faecalis VRE-1 (ATCC™ 29212), Enterococcus faecalis VRE-2 (ATCC™ 51299), Escherichia coli (ATCC™ 25922), Streptococcus pneumoniae (LSPQ 3412) and Streptococcus pneumoniae PenR (LSPQ 3349). Indicator strains preparation and MIC determination were performed according to the National Committee for Clinical Laboratory Standards (NCCLS) guideline M7-A5 Methods for Dilution Antimicrobial Susceptibility Tests for Bacteria That Grow Aerobically; Approved Standard-Fifth Edition. (NCCLS document M7-A5, ISBN 1-56238-394-9; NCCLS, 940 West Valley Road, Suite 1400, Wayne, Pa. 19087-1898 USA), the content of which is incorporated herein by reference. MIC determination against C. difficile was performed according to National Committee for Clinical Laboratory Standards (NCCLS) guideline M11-A5 Methods for Antimicrobial Susceptibility Testing of Anaerobic Bacteria.

Compounds 1 and 2 were prepared as 100× stock solutions in DMSO, with concentrations ranging from 3.2 mg/mL to 0.003 mg/mL (a two-fold dilution series over 11 points). An aliquot of each 100× stock solution was diluted 50-fold in test medium described below to give a set of eleven (11) 2× solutions. 50 μL of each of the eleven 2× solutions was aliquoted into the corresponding well of a 12-well row, with the final well reserved for medium alone control.

Vancomycin (Sigma™), used as positive control compound, was prepared as 2× stock solutions in Mueller-Hinton test medium ranging from 64 μg/mL to 0.06 μg/ml (a two-fold dilution series over 11 points). An aliquot of 50 μL corresponding to each concentration (at 2×) was then transferred to 96-well microplates to obtain a series of eleven two-fold dilutions.

An isolated colony of each of the indicator strains was used to inoculate tubes containing 2 mL of test medium. MH (Mueller-Hinton) test medium was used for Staphylococcus aureus (ATCC™ 6538P), Staphylococcus aureus MRS3 (ATCC™ 700699), Escherichia coli (ATCC™ 25922) indicator strains. BHI+ (Brain Heart Infusion broth supplemented with 5 mM CaCl₂) test medium was used for Enterococcus faecalis VRE-1 (ATCC™ 29212) and Enterococcus faecalis VRE-2 (ATCC™ 51299) indicator strains. MH test medium (+2% lysed horse blood) was used for Streptococcus pneumoniae cell lines. Brucella medium supplemented with hemin, vitamin K1 and 5% lysed horse blood was used for Clostridium difficile (ATCC™ 9689). Inoculum density for each indicator strain was adjusted to OD₆₀₀=0.1 in 5 ml 0.85% saline, then further diluted 1/100 in appropriate medium. 50 μL of the final dilution (in test medium) of each indicator strain was added to each well of a 12-well row. This brings the final dilution of the test article or control compound in solution to 1×. The final inoculum was approximately 5×10⁵ CFU/mL.

The indicator strains were incubated with 11 concentrations of each of Compound 1, Compound 2, Vancomycin (Sigma™ ) control compound and one media alone control. For MIC determination, assay plates were incubated at 35° C. for 16 to 20 hours. The MIC for each indicator was assessed as the lowest concentration of compound resulting in total absence of growth and is shown below.

TABLE 7
Antibacterial activity of Compounds 1 and 2, MIC (μg/mL)

| Strain Information | | Test Compound | | |
|---|---|---|---|---|
| Strain | Source | 1 | 2 | Vancomycin |
| Staphylococcus aureus | ATCC ™ 6538P | 0.016 | 0.0625 | 1 |
| Staphylococcus aureus MRS3 | ATCC ™ 700699 | 0.016 | 0.125 | 4 |
| Enterococcus faecalis VRE-1 | ATCC ™ 29212 | 0.063 | 1 | 8/4* |
| Enterococcus faecalis VRE-2 | ATCC ™ 51299 | 0.125 | 1 | 32 |
| Streptococcus pnuemoniae | LSPQ 3412 | 0.5-1 | 1 | 0.5 |
| Streptococcus pneumoniae PenR | LSPQ 3349 | 4-16 | 1 | 0.5 |
| Clostridium difficile | ATCC ™ 9689 | 0.06 | 2 | 0.5 |

*when tested as control with Compound 2

Compounds 1 and 2, as shown in Table 7, proved to be very potent antibacterial agents against several gram positive strains, including Enterococci and Staphylococci strains. They both exhibited activities similar to Vancomycin against S. pneumoniae strains. Compound 1 also exhibited very potent antibacterial activity against Clostridium difficile. Compounds 1 and 2 were inactive against Escherichia coli.

B. In vitro Antifungal Activity of Compound 1:

Compound 1 exhibited antifungal activity (MIC: 8 μg/mL after 24 hours) against Saccharomyces cerevisiae FHCRC-50014 and FHCRC-50514, where Fluconazole had MICs of 8 μg/mL and 0.25-0.5 μg/mL respectively, as control.

Testing of antifungal activity was performed according to NCCLS method (NCCLS. Reference Method for Broth Dilution Antifungal Susceptibility Testing of Yeasts; Approved Standard. NCCLS document M27-A (ISBN 1-56238-328-0). NCCLS, 940 West Valley Road, Suite 1400, Wayne, Pa. 19087-1898 USA; www.nccls.org.). Compound 1 was prepared as 100× stock solutions in DMSO, with concentrations ranging from 3.2 mg/mL to 0.003 mg/mL (a two-fold dilution series over 11 points). An aliquot of each 100× stock solution was diluted 50-fold in test medium described below to give a set of eleven (11) 2× solutions. 50 μL of each of the eleven 2× solutions was aliquoted into the corresponding well of a 12-well row, with the final well reserved for medium alone control. Inoculum density for each indicator strain was adjusted to OD₆₀₀=0.1 in 5 mL 0.85% saline, then further diluted 1/1000 in YPD medium. 50 μL of the final dilution (in test medium) of each indicator strain was added to each well of a 12-well row. This brings the final dilution of the test article or control compound in solution to 1×. The final inoculum was approximately 5×10⁴ CFU/ml.

Bacterial strains Streptococcus pneumoniae LSPQ 3412 and LSPQ 3349 were obtained from the “Laboratoire de Sante Publique du Quebec”. Saccharomyces cerevisiae FHCRC-50014 and FHCRC-50514 were obtained from the “Fred Hutchison Cancer Research Center”.

### Example 7

### In vivo Antimicrobial Activity of Compound 1

In vivo efficacy studies were performed on ICR mice (supplied by Charles River Laboratories, Wilmington, Mass.) having a bodyweight of 20±2 g. Mice (38) were inoculated i.p. with an LD₉₀₋₁₀₀ dose (1×10⁶ CFU/mouse) of Staphylococcus aureus (Smith strain, ATCC™ 13709) suspended in 0.5 mL of BHI containing 5% mucin. Compound 1 at doses 0.1, 0.5, 1 and 5 mg/kg (formulated in 1% SDS for a total volume of 5 μL/g of weight) was administered i.p. one hour post infection (5 mice for each concentration). Another group of 5 mice was treated i.v. with a 10 mg/kg dose of Vancomycin. Finally, a group of 3 mice was administered vehicle only. Mortality was recorded for 7 days. Compound 1 showed a median effective dose (ED₅₀) lower than 0.1 mg/kg when administered i.p.

### Example 8

### Synthesis of Compound 2 by Esterification of Compound 1

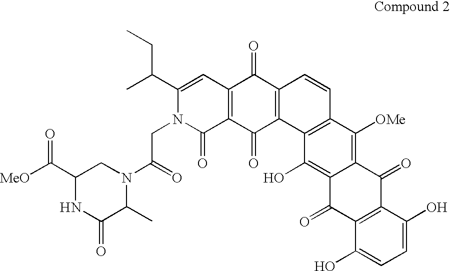

Excess sodium carbonate is added to Compound 1 in dimethyl sulfoxide. lodomethane (1.1 equivalent) is slowly added and the reaction is stirred at room temperature for two hours. The reaction is acidified with trifluoroacetic acid, filtered and purified by HPLC as shown in Procedure 3 of Example 2. Pure Compound 2 is obtained by pooling and concentrating the appropriate eluate fractions.

### Example 9

### Synthesis of Compound 13 by Etherification of Compound 2

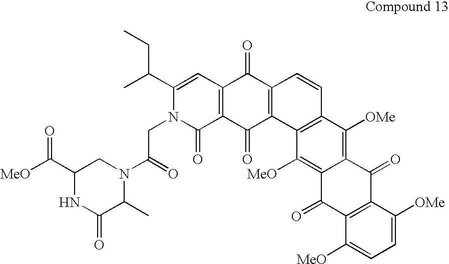

At least 3 molar equivalents of a 0.1 M solution of sodium hydroxide in methanol (Fisher Chemicals) are added to Compound 2 in methanol. At least 3 molar equivalents of dimethyl sulfate are added and the reaction stirred at room temperature for 24 hours. The reaction is monitored and additional sodium hydroxide in methanol and dimethyl sulfate are added and the reaction stirred until completion. The reaction is concentrated in vacuo. The crude residue is purified by HPLC as shown in Procedure 3 of Example 2. Pooling and concentration of the appropriate fractions give pure Compound 13.

### Example 10

### Synthesis of Compound 24, by Esterification of Compound 1

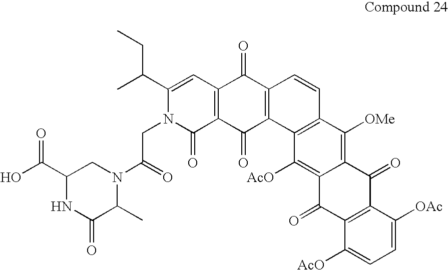

Acetic anhydride (4.5 equivalents) is added dropwise to a solution of Compound 1 in acetonitrile and pyridine (9:1) and the reaction stirred at room temperature until completion. The solvent is removed under vacuum and the crude residue is purified by HPLC as shown in Procedure 3 of Example 2. Pooling and concentration of the appropriate fractions give pure Compound 24.

### Example 11

### Synthesis of Compound 11, by Amidation of Compound 24

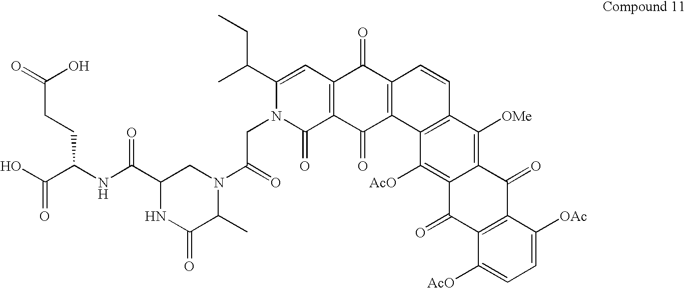

EDC (1-(3-dimethylaminopropyl)-3-diisopropylethylcarbodiimide hydrochloride) is added to a solution of Compound 24 in dichloromethane. DIPEA (N,N-diisopropylethylamine), L-Glutamic acid di-benzyl ester and a catalytic amount of DMAP (4-(dimethylamino)pyridine) are added and the reaction stirred overnight. The reaction mixture is washed three times with a 1 N hydrochloric acid aqueous solution. Organic layer is separated, dried over magnesium sulfate, filtered and concentrated in vacuo. The crude residue is dissolved in ethanol and treated with a catalytic amount of Pd/C (palladium on charcoal) under hydrogen atmosphere until completion. The suspension is filtered through a celite pad under inert atmosphere and the solution concentrated in vacuo. The crude residue is purified by HPLC as shown in Procedure 3.of Example 2. Pooling and concentration of the appropriate fractions give pure Compound 11.

### Example 12

### Synthesis of Compound 27, by Esterification of Compound 1

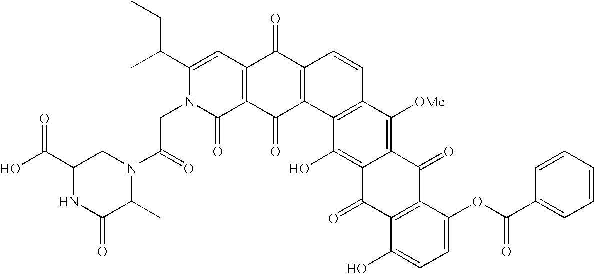

### Compound 27

Benzoyl chloride (1 molar equivalent) is added dropwise to a 0° C. solution of Compound 1 in acetonitrile and pyridine (9:1). The mixture is stirred at room temperature until completion. The solvent is removed under vacuum and the crude residue is purified by HPLC as shown in Procedure 3 of Example 2. Pooling and concentration of the appropriate fractions give pure Compound 27.

### Example 13

### Synthesis of Compound 28, by Demethylation of Compound 1

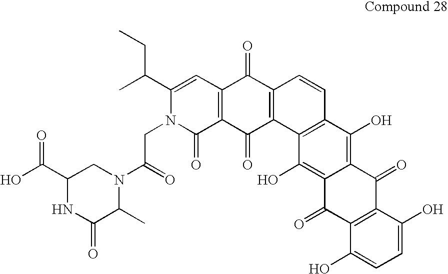

A. Procedure 1

Boron tribromide (1 molar equivalent) is added dropwise to a −78° C. solution of Compound 1 in tetrahydrofuran. The mixture is stirred until completion and water is added dropwise. The reaction mixture is neutralized and extracted with 3 volumes of ethyl acetate. Organic layers are combined and dried over magnesium sulfate, filtered and concentrated in vacuo. The crude residue is purified by HPLC as shown in Procedure 3 of Example 2. Pooling and concentration of the appropriate fractions give pure Compound 28.

B. Procedure 2

Lithium chloride (1 eq) is added to a solution of Compound 1 in DMF (N,N-dimethylformamide) and the reaction mixture stirred under reflux until completion. The reaction mixture is allowed to cool to room temperature and concentrated in vacuo. The crude residue is purified by HPLC as shown in Example Procedure 3 of 2. Pooling and concentration of the appropriate fractions give pure Compound 28.

### Example 14

### Synthesis of Compound 29, by Reduction of Compound 1

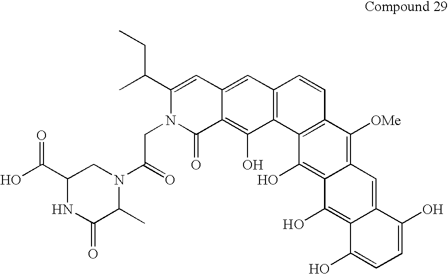

Compound 1 is reduced to produce Compound 29 according to the conditions described in J. March, “Advanced Organic Chemistry”, 4^{th} Edition, John Wiley & Sons, New York (1992), at page 1210 (and in Meyer et al., Org. Synth. I, 60). The crude residue obtained from working up the reaction is purified by HPLC as shown in Procedure 3 of Example 2. Pooling and concentration of the appropriate fractions give pure Compound 29 or one of its tautomeric forms.

### Example 15

### Synthesis of Compound 35, by Reduction of Compound 1

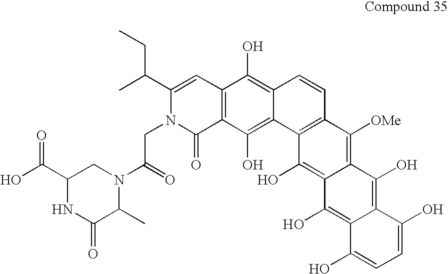

An excess of sodium borohydride is added to a −78° C. solution of Compound 1 in tetrahydrofuran. The reaction is stirred until completion. The reaction temperature is raised to 0° C. if needed to attain completion. A saturated aqueous ammonium chloride solution is added to the reaction mixture and extracted with 3 portions of ethyl acetate. Organic layers are combined, dried over magnesium sulfate and concentrated in vacuo. The crude residue obtained from working up the reaction is purified by HPLC as shown in Procedure 3 of Example 2. Pooling and concentration of the appropriate fractions give pure Compound 35.

### Example 15

### Synthesis of Base Addition Salts of Compound 1

Base addition salts of Compound 1, for example ammonium, sodium and potassium salts are prepared by reacting Compound 1 with the corresponding base. For example ammonia (bubbled in a solvent such as acetonitrile, and concentrated in vacuo), or by treating with one molar equivalent of an aqueous ammonium hydroxide. The aqueous solution is concentrated in vacuo, or freeze-dried to give the ammonium salt. Sodium and potassium salts are prepared by reacting Compound 1 with one molar equivalent of the corresponding base, e.g. aqueous sodium or potassium bicarbonate, or sodium or potassium hydroxide. Aqueous solutions of the salt formed are freeze-dried to give the desired base addition salt.

All patents, patent applications, and published references cited herein are hereby incorporated by reference in their entirety. While this invention has been particularly shown and described with reference to preferred embodiments thereof, it will be understood by those skilled in the art that various changes in form and details may be made therein without departing from the scope of the invention encompassed by the appended claims.

1. A compound of Formula I, or a tautomer, or a pharmaceutically acceptable salt or prodrug thereof: 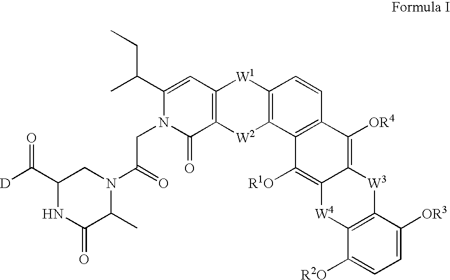
wherein,
D is selected from the group consisting of OH, —OR⁵ and —NR⁶R⁷;
W¹ and W² are each C(O); or W¹ and W² are each C(OR⁸) when taken together with their adjacent carbon atoms to form an aromatic ring; or one of W¹ and W² is C(OR⁸) and the other is C(H) when taken together with their adjacent carbon atoms to form an aromatic ring;
W³ and W⁴ are each C(O); or W³ and W⁴ are each C(OR⁸) when taken together with their adjacent carbon atoms to form an aromatic ring; or one of W³ and W⁴ is C(OR⁸) and the other is C(H) when taken together with their adjacent carbon atoms to form an aromatic ring;
R¹, R², R³, R⁴ and R⁸ are each independently selected from H, R⁹ and C(O)R¹⁰;
R⁵ is selected from the group consisting of C₁₋₁₀alkyl, C₂₋₁₀alkenyl, C₂₋₁₀alkynyl, C₃₋₁₀cycloalkyl, C₃₋₁₀heterocycloalkyl, C₅₋₁₀aryl and C₅₋₁₀heteroaryl;
R⁶ is selected from the group consisting of H, C₁₋₁₀alkyl, C₂₋₁₀alkenyl and C₂₋₁₀alkynyl;
R⁷ is selected from the group consisting of H, C₁₋₁₀alkyl, C₂₋₁₀alkenyl, C₂₋₁₀alkynyl, C₅₋₁₀aryl, C₅₋₁₀heteroaryl, C₃₋₁₀cycloalkyl and C₃₋₁₀heterocycloalkyl; or the group —NR⁶R⁷ is an N-coupled amino acid;
R⁹ is selected from the group consisting of C₁₋₁₀alkyl, C₂₋₁₀alkenyl, C₂₋₁₀alkynyl, C₃₋₁₀cycloalkyl and C₃₋₁₀heterocycloalkyl;
R¹⁰ is selected from the group consisting of H, C₁₋₁₀alkyl, C₂₋₁₀alkenyl, C₂₋₁₀alkynyl, C₅₋₁₀aryl, C₅₋₁₀heteroaryl, C₃₋₁₀cycloalkyl and C₃₋₁₀heterocycloalkyl; or —C(O)R¹⁰ is a C-coupled amino acid;
wherein, when any of R¹, R², R³, R⁴, R⁵, R⁶, R⁷, R⁸, R⁹ and R¹⁰ comprises an alkyl, alkenyl, alkynyl, aryl, heteroaryl, cycloalkyl, or heterocycloalkyl group, then the alkyl, alkenyl, alkynyl, aryl, heteroaryl, cycloalkyl, or heterocycloalkyl group is optionally substituted with substituents selected from acyl, amino, acylamino, acyloxy, carboalkoxy, carboxy, carboxyamido, cyano, halo, hydroxyl, nitro, thio, C₁₋₆alkyl, C₂₋₇alkenyl, C₂₋₇alkynyl, C₃₋₁₀cycloalkyl, C₃₋₁₀heterocycloalkyl, C₆₋₁₀aryl, C₅₋₁₀heteroaryl, alkoxy, aryl sulfinyl, sulfonyl, oxo, guanidino and formyl.

2. The compound of claim 1, wherein said compound is a compound of Formula II, or a tautomer, or a pharmaceutically acceptable salt or prodrug thereof: 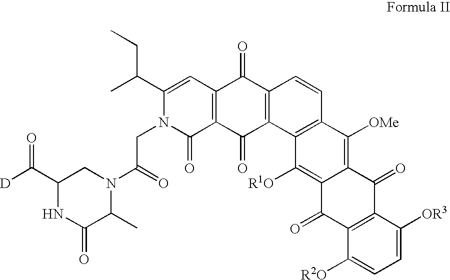
wherein,
D is selected from the group consisting of OH, —OR⁵ and —NR⁶R⁷;
R¹, R² and R³ are each independently selected from H, R⁹ and C(O)R¹⁰;
R⁵is selected from the group consisting of C₁₋₁₀alkyl, C₂₋₁₀alkenyl, C₂₋₁₀alkynyl, C₃₋₁₀cycloalkyl, C₃₋₁₀heterocycloalkyl, C₅₋₁₀aryl and C₅₋₁₀heteroaryl;
R⁶ is selected from the group consisting of H, C₁₋₁₀alkyl, C₂₋₁₀alkenyl and C₂₋₁₀alkynyl;
R⁷is selected from the group consisting of C₁₋₆alkyl, C₅₋₁₀aryl, C₅₋₁₀heteroaryl, C₃₋₁₀cycloalkyl and C₃₋₁₀heterocycloalkyl; or the group —NR⁶R⁷ is an N-coupled amino acid;
R⁹ is selected from the group consisting of C₁₋₁₀alkyl, C₂₋₁₀alkenyl, C₂₋₁₀alkynyl, C₃₋₁₀cycloalkyl and C₃₋₁₀heterocycloalkyl;
R¹⁰ is selected from the group consisting of H, C₁₋₁₀alkyl, C₂₋₁₀alkenyl, C₂₋₁₀alkynyl, C₅₋₁₀aryl, C₅₋₁₀heteroaryl, C₃₋₁₀cycloalkyl and C₃₋₁₀heterocycloalkyl; or —C(O)R¹⁰ is a C-coupled amino acid
wherein, when any of R¹, R², R³, R⁵, R⁶, R⁷, R⁹ and R¹⁰ comprises an alkyl, alkenyl, alkynyl, aryl, heteroaryl, cycloalkyl, or heterocycloalkyl group, then the alkyl, alkenyl, alkynyl, aryl, heteroaryl, cycloalkyl, or heterocycloalkyl group is optionally substituted with substituents selected from acyl, amino, acylamino, acyloxy, carboalkoxy, carboxy, carboxyamido, cyano, halo, hydroxyl, nitro, thio, C₁₋₆alkyl, C₂₋₇alkenyl, C₂₋₇alkynyl, C₃₋₁₀cycloalkyl, C₃₋₁₀heterocycloalkyl, C₆₋₁₀aryl, C₅₋₁₀heteroaryl, alkoxy, aryloxy, sulfinyl, sulfonyl, oxo, guanidino and formyl.

3. The compound of claim 1, wherein said compound is Compound 1 having the formula: 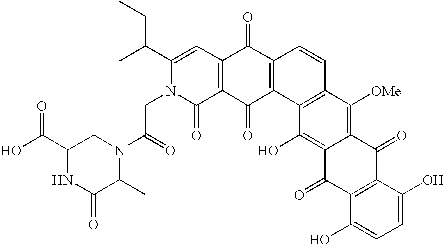
or a pharmaceutically acceptable salt or prodrug thereof.

4. The compound of claim 1, wherein said compound is Compound 2 having the formula: 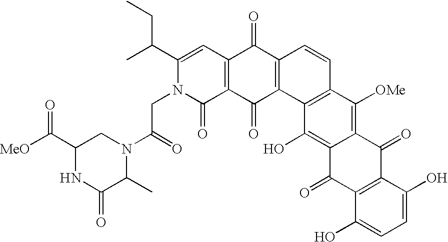
or a tautomer, or a pharmaceutically acceptable salt or prodrug thereof.

5. The compound of claim 1, wherein said compound is selected Compounds 1 to 35, or a tautomer, or a pharmaceutically acceptable salt or prodrug thereof:

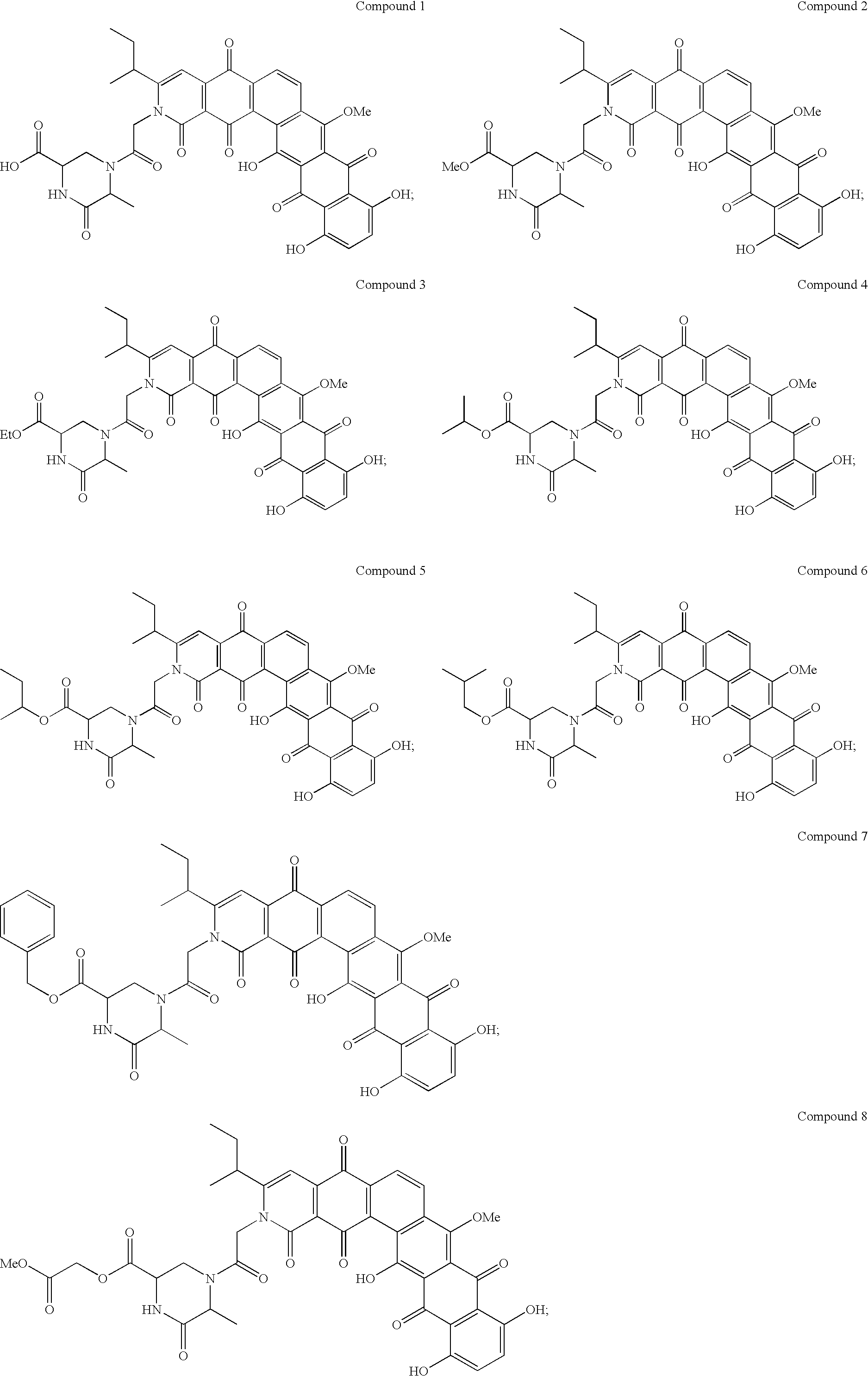

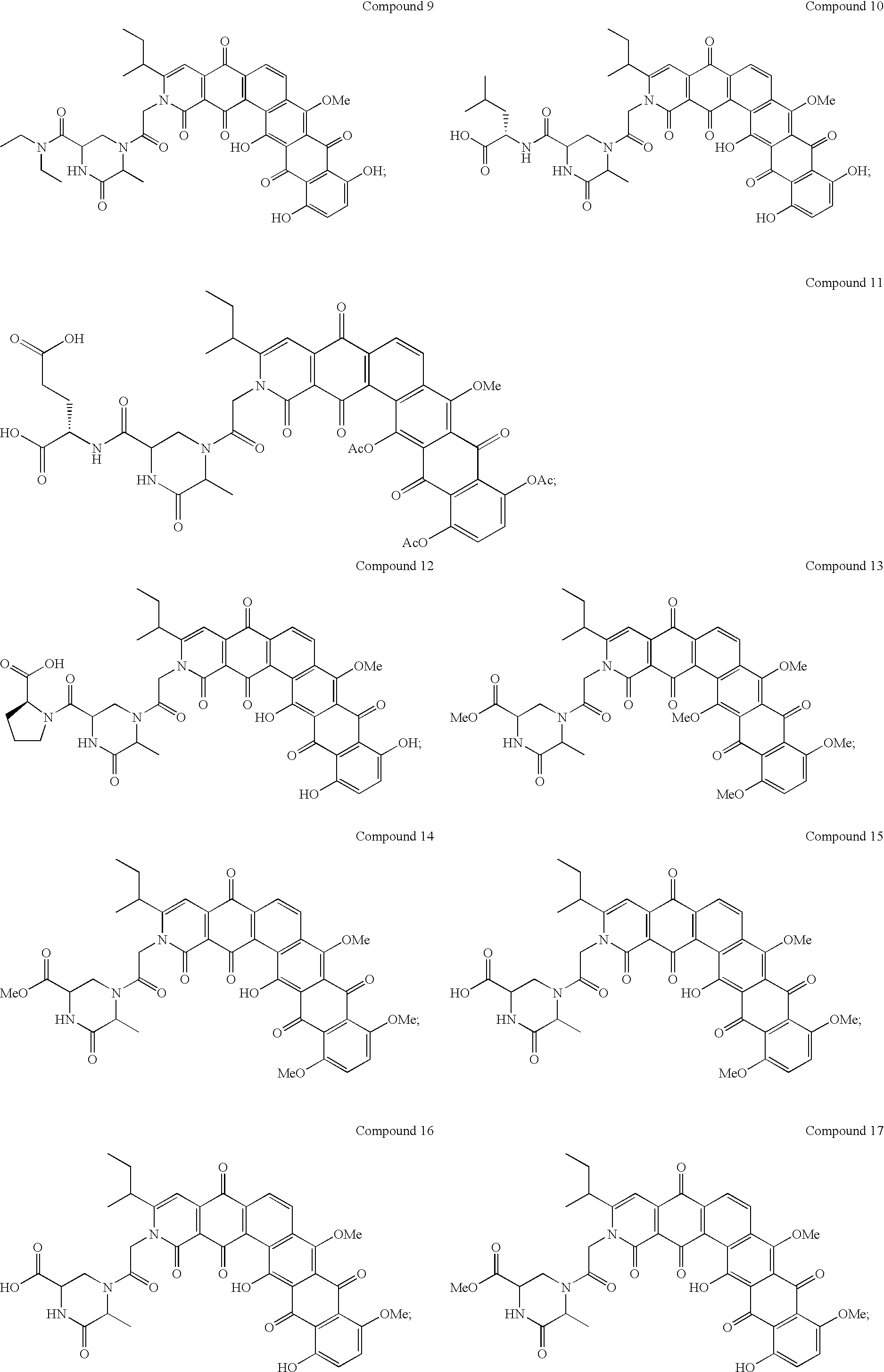

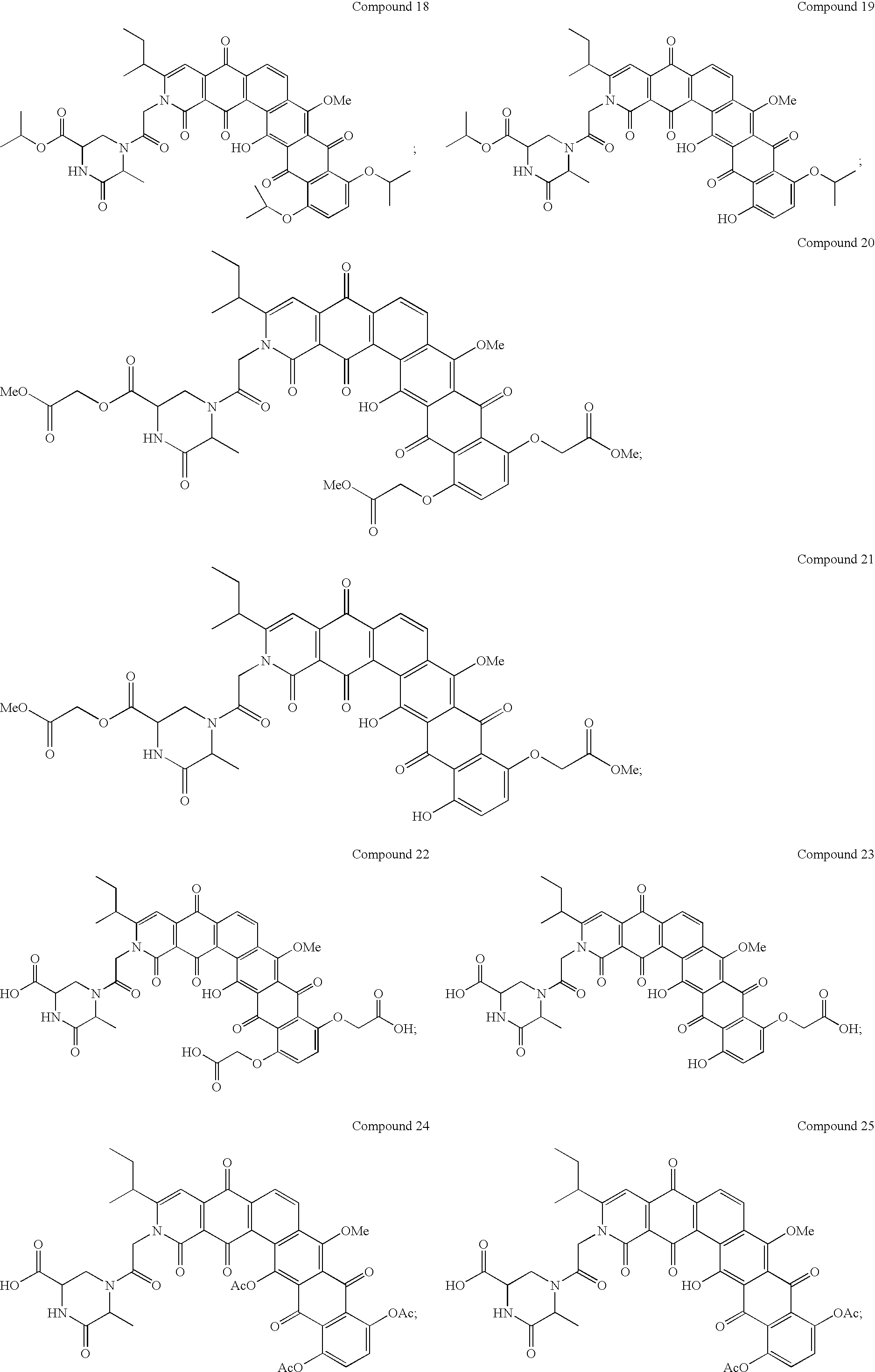

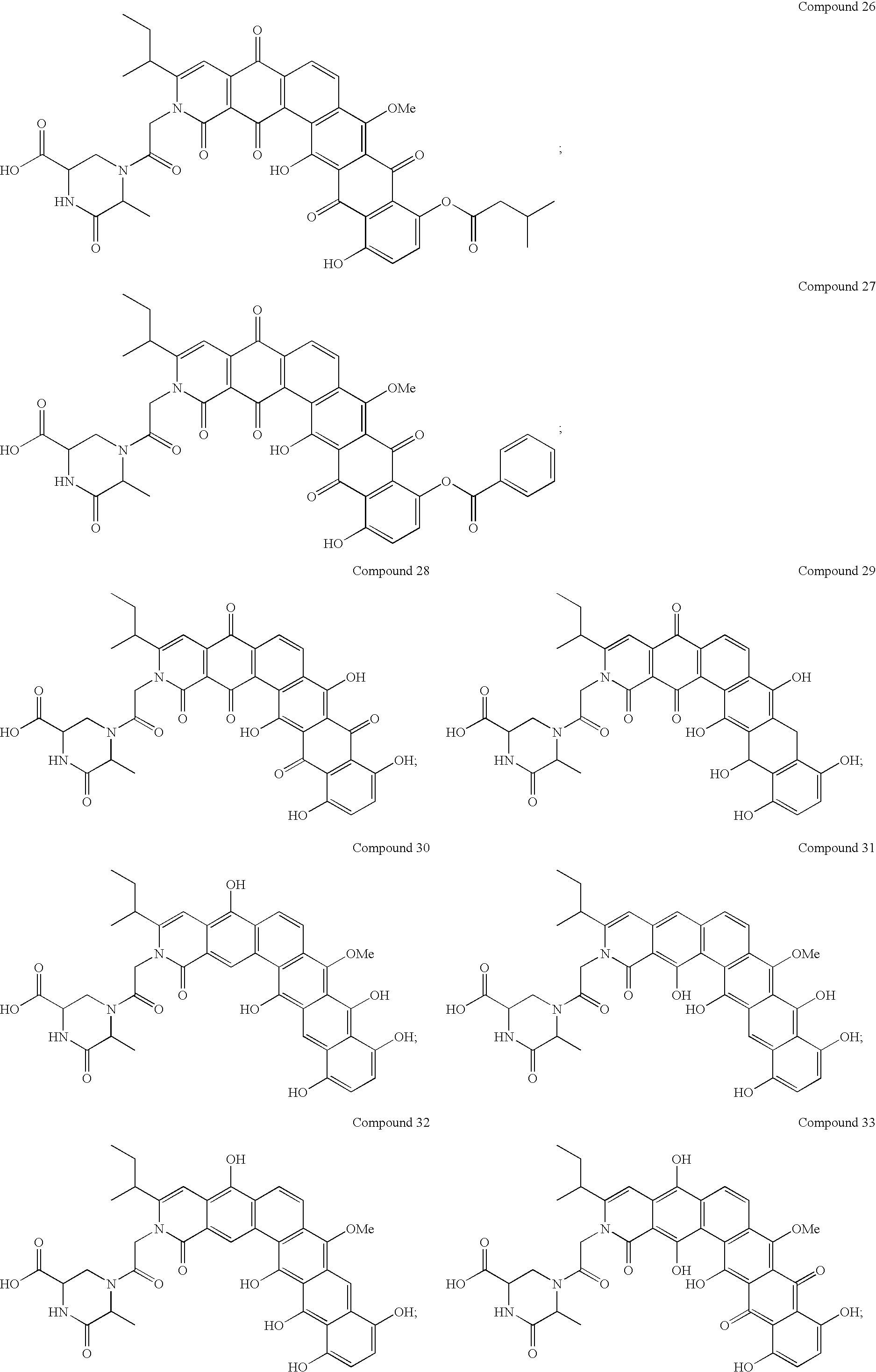

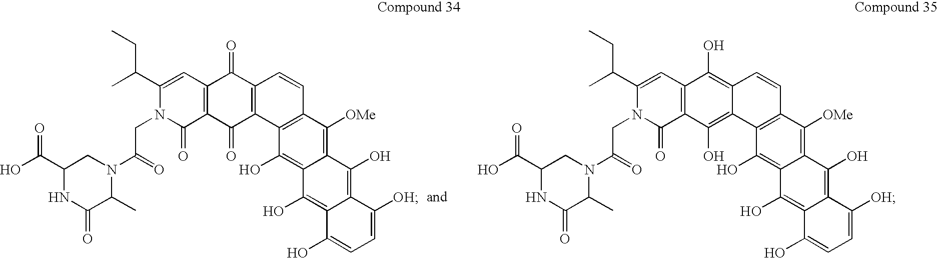

6. A compound of claim 1, obtained by a method selected from:
a) the method comprising the steps of cultivating a Micromonospora strain under aerobic conditions in a nutrient medium comprising at least one source of carbon atoms and at least one source of nitrogen atoms, and isolating a compound from said bacteria; and
b) the method of paragraph a), wherein the compound obtained from cultivation and isolation is further chemically modified.

7. The compound of claim 6, wherein said Micromonospora strain is Micromonospora echinospora ssp. Challisensis NRRL-1 2255, or a mutant, or a variant thereof.

8. A process of producing a compound of claim 1, said process comprising the steps of:
a) cultivating a Micromonospora strain under aerobic conditions in a nutrient medium comprising at least one source of carbon atoms and at least one source of nitrogen atoms; and
b) isolating a compound from said bacteria.

9. The process of claim 8, said process further comprising the step of chemically modifying the compound obtained from isolation step (b).

10. The process of claim 8, wherein said Micromonospora strain is Micromonospora echinospora ssp. Challisensis NRRL-1 2255 or a mutant, or a variant thereof.

11. A pharmaceutical composition comprising a therapeutically effective amount of a compound of claim 1, together with a pharmaceutically acceptable carrier.

12. A pharmaceutical composition comprising a therapeutically effective amount of a compound of claim 2, together with a pharmaceutically acceptable carrier.

13. A pharmaceutical composition comprising a therapeutically effective amount of a compound of claim 3, together with a pharmaceutically acceptable carrier.

14. A pharmaceutical composition comprising a therapeutically effective amount of a compound of claim 4, together with a pharmaceutically acceptable carrier.

15. A pharmaceutical composition comprising a therapeutically effective amount of a compound of claim 5, together with a pharmaceutically acceptable carrier.

16. A method of treating a neoplastic condition in a mammal, comprising administering to a mammal in need of such treatment, a therapeutically effective amount of a compound of claim 1.

17. A method of treating a neoplastic condition in a mammal, comprising administering to a mammal in need of such treatment, a therapeutically effective amount of a compound of claim 3.

18. A method of treating a neoplastic condition in a mammal, comprising administering to a mammal in need of such treatment, a therapeutically effective amount of a compound of claim 4.

19. A method of treating a neoplastic condition in a mammal, comprising administering to a mammal in need of such treatment, a therapeutically effective amount of a pharmaceutical composition of claim 11.

20. A method of treating a neoplastic condition in a mammal, comprising administering to a mammal in need of such treatment, a therapeutically effective amount of a pharmaceutical composition of claim 13.

21. A method of treating a neoplastic condition in a mammal, comprising administering to a mammal in need of such treatment, a therapeutically effective amount of a pharmaceutical composition of claim 14.

22. A method of treating a neoplastic condition in a mammal, comprising administering to a mammal in need of such treatment, a therapeutically effective amount of a pharmaceutical composition of claim 15.

23. A method of treating a bacterial infection in a mammal, comprising administering to a mammal in need of such treatment, a therapeutically effective amount of a compound of claim 1.

24. A method of treating a bacterial infection in a mammal, comprising administering to a mammal in need of such treatment, a therapeutically effective amount of a compound of claim 3.

25. A method of treating a bacterial infection in a mammal, comprising administering to a mammal in need of such treatment, a therapeutically effective amount of a compound of claim 4.
